SEQUENCE LISTING (62 sequences)

SEQ ID NO: 1 - protein, 470 aa, synthetic construct
  source 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, synthetic construct
  source 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, synthetic construct
  source 1..5: note = CDRH1 of heavy chain of anti-TROP2 antibody
TAGMQ

SEQ ID NO: 4 - protein, 17 aa, synthetic construct
  source 1..17: note = CDRH2 of heavy chain of anti-TROP2 antibody
WINTHSGVPKYAEDFKG

SEQ ID NO: 5 - protein, 12 aa, synthetic construct
  source 1..12: note = CDRH3 of heavy chain of anti-TROP2 antibody
SGFGSSYWYFDV

SEQ ID NO: 6 - protein, 11 aa, synthetic construct
  source 1..11: note = CDRL1 of light chain of anti-TROP2 antibody
KASQDVSTAVA

SEQ ID NO: 7 - protein, 7 aa, synthetic construct
  source 1..7: note = CDRL2 of light chain of anti-TROP2 antibody
SASYRYT

SEQ ID NO: 8 - protein, 9 aa, synthetic construct
  source 1..9: note = CDRL3 of light chain of anti-TROP2 antibody
QQHYITPLT

SEQ ID NO: 9 - protein, 121 aa, synthetic construct
  source 1..121: note = Variable region of heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 10 - protein, 109 aa, synthetic construct
  source 1..109: note = Variable region of light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 11 - protein, 450 aa, synthetic construct
  source 1..450: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 451 aa, synthetic construct
  source 1..451: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  source 1..214: note = Light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 450 aa, synthetic construct
  source 1..450: note = Heavy chain of humanized anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, synthetic construct
  source 1..214: note = Light chain of humanized anti-HER2 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 8 aa, synthetic construct
  source 1..8: note = CDRH1 of heavy chain of humanized anti-HER2 antibody
GFNIKDTY

SEQ ID NO: 17 - protein, 8 aa, synthetic construct
  source 1..8: note = CDRH2 of heavy chain of humanized anti-HER2 antibody
IYPTNGYT

SEQ ID NO: 18 - protein, 13 aa, synthetic construct
  source 1..13: note = CDRH3 of heavy chain of humanized anti-HER2 antibody
SRWGGDGFYAMDY

SEQ ID NO: 19 - protein, 6 aa, synthetic construct
  source 1..6: note = CDRL1 of light chain of humanized anti-HER2 antibody
QDVNTA

SEQ ID NO: 20 - protein, 7 aa, synthetic construct
  source 1..7: note = Sequence comprising CDRL2 of light chain of humanized anti-HER2 antibody
SASFLYS

SEQ ID NO: 21 - protein, 9 aa, synthetic construct
  source 1..9: note = CDRL3 of light chain of humanized anti-HER2 antibody
QQHYTTPPT

SEQ ID NO: 22 - protein, 120 aa, synthetic construct [sequence omitted]

SEQ ID NO: 23 - protein, 107 aa, synthetic construct [sequence omitted]

SEQ ID NO: 24 - protein, 449 aa, synthetic construct
  source 1..449: note = Heavy chain of humanized anti-HER2 antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 5 aa, synthetic construct
  source 1..5: note = Synthetic peptide
DYGMH

SEQ ID NO: 26 - protein, 17 aa, synthetic construct
  source 1..17: note = Synthetic peptide
YISSGSYTIYSADSVKG

SEQ ID NO: 27 - protein, 13 aa, synthetic construct
  source 1..13: note = Synthetic peptide
RAPNSFYEYYFDY

SEQ ID NO: 28 - protein, 16 aa, synthetic construct
  source 1..16: note = Synthetic peptide
SASSKHTNLYWSRHMY

SEQ ID NO: 29 - protein, 7 aa, synthetic construct
  source 1..7: note = Synthetic peptide
LTSNRAT

SEQ ID NO: 30 - protein, 9 aa, synthetic construct
  source 1..9: note = Synthetic peptide
QQWSSNPFT

SEQ ID NO: 31 - protein, 122 aa, synthetic construct
  source 1..122: note = Synthetic peptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSS

SEQ ID NO: 32 - protein, 452 aa, synthetic construct
  source 1..452: note = Synthetic peptide
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 112 aa, synthetic construct
  source 1..112: note = Synthetic peptide
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIK

SEQ ID NO: 34 - protein, 219 aa, synthetic construct
  source 1..219: note = Synthetic peptide
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 5 aa, synthetic construct
  source 1..5: note = Synthetic peptide
SYAMH

SEQ ID NO: 36 - protein, 17 aa, synthetic construct
  source 1..17: note = Synthetic peptide
VISYAGEVKYYADSVKG

SEQ ID NO: 37 - protein, 12 aa, synthetic construct
  source 1..12: note = Synthetic peptide
DPLPLHYYGMDV

SEQ ID NO: 38 - protein, 13 aa, synthetic construct
  source 1..13: note = Synthetic peptide
SGSSSNIGRRPVN

SEQ ID NO: 39 - protein, 7 aa, synthetic construct
  source 1..7: note = Synthetic peptide
SQNQRPS

SEQ ID NO: 40 - protein, 11 aa, synthetic construct
  source 1..11: note = Synthetic peptide
AVWDDIGRVLQ

SEQ ID NO: 41 - protein, 121 aa, synthetic construct
  source 1..121: note = Synthetic peptide
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSS

SEQ ID NO: 42 - protein, 451 aa, synthetic construct
  source 1..451: note = Synthetic peptide
QVQLVESGGGVVQPGRSLRLSCAASGFTFSSYAMHWVRQAPGKGLEWVAVISYAGEVKYYADSVKGRFTISRDNSKNTLYLQMNSLRAEDTAVYYCARDPLPLHYYGMDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 110 aa, synthetic construct
  source 1..110: note = Synthetic peptide
QSVLTQPPSASGTPGQRVTISCSGSSSNIGRRPVNWYQQLPGTAPKLLIYSQNQRPSGVPDRFSGSQSGTSASLAISGLQSEDEADYFCAVWDDIGRVLQLGGGTQLTVL

SEQ ID NO: 44 - protein, 216 aa, synthetic construct
  source 1..216: note = Synthetic peptide
QSVLTQPPSASGTPGQRVTISCSGSSSNIGRRPVNWYQQLPGTAPKLLIYSQNQRPSGVPDRFSGSQSGTSASLAISGLQSEDEADYFCAVWDDIGRVLQLGGGTQLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 45 - protein, 219 aa, synthetic construct; identical to SEQ ID NO: 34
  source 1..219: note = DuetMab PD-1 (LO115) Light Chain

SEQ ID NO: 46 - protein, 452 aa, synthetic construct; identical to SEQ ID NO: 32
  source 1..452: note = DuetMab PD-1 (LO115) Heavy Chain

SEQ ID NO: 47 - protein, 213 aa, synthetic construct
  source 1..213: note = DuetMab CTLA-4 Light Chain
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 48 - protein, 455 aa, synthetic construct
  source 1..455: note = DuetMab CTLA-4 Heavy Chain
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 18 aa, synthetic construct
  source 1..18: note = PD-1 (LO115) LC - CDR1
SASSKHTNLYWSRHMYWY

SEQ ID NO: 50 - protein, 6 aa, synthetic construct
  source 1..6: note = PD-1 (LO115) LC - CDR2
TSNRAT

SEQ ID NO: 51 - protein, 7 aa, synthetic construct
  source 1..7: note = PD-1 (LO115) LC - CDR3
QQWSSNP

SEQ ID NO: 52 - protein, 10 aa, synthetic construct
  source 1..10: note = PD-1 (LO115) HC - CDR1
GFTFSDYGMH

SEQ ID NO: 53 - protein, 17 aa, synthetic construct
  source 1..17: note = PD-1 (LO115) HC - CDR2
YISSGSYTIYSADSVKG

SEQ ID NO: 54 - protein, 13 aa, synthetic construct
  source 1..13: note = PD-1 (LO115) HC - CDR3
RAPNSFYEYYFDY

SEQ ID NO: 55 - protein, 11 aa, synthetic construct
  source 1..11: note = CTLA-4 LC - CDR1
RASQSINSYLD

SEQ ID NO: 56 - protein, 7 aa, synthetic construct
  source 1..7: note = CTLA-4 LC - CDR2
AASSLQS

SEQ ID NO: 57 - protein, 7 aa, synthetic construct
  source 1..7: note = CTLA-4 LC - CDR3
QQYYSTP

SEQ ID NO: 58 - protein, 10 aa, synthetic construct
  source 1..10: note = CTLA-4 HC - CDR1
GFTFSSYGMH

SEQ ID NO: 59 - protein, 17 aa, synthetic construct
  source 1..17: note = CTLA-4 HC - CDR2
VIWYDGSNKYYADSVKG

SEQ ID NO: 60 - protein, 16 aa, synthetic construct
  source 1..16: note = CTLA-4 HC - CDR3
DPRGATLYYYYYGMDV

SEQ ID NO: 61 - protein, 4 aa, synthetic construct
  SITE 1: note = Antibody conjugated 6-(2,5-dioxo-2,5-dihydro-1H-pyrrol-1-yl)hexanoyl glycine
  SITE 4: note = (2-{[(1S,9S)-9-ethyl-5-fluoro-9-hydroxy-4-methyl-10 ,13-dioxo-2,3,9,10,13,15-hexahydro-1H,12H-benzo[de]pyrano[ 3',4':6,7]indolizino[1,2-b]quinolin-1-yl]amino}-2-oxoethox y)methylamino glycine
GGFG

SEQ ID NO: 62 - protein, 4 aa, synthetic construct
  SITE 1: note = 6-(2,5-dioxo-2,5-dihydro-1H-pyrrol-1-yl)hexanoyl glycine
  SITE 4: note = (2-{[(1S,9S)-9-ethyl-5-fluoro-9-hydroxy-4-methyl-10 ,13-dioxo-2,3,9,10,13,15-hexahydro-1H,12H-benzo[de]pyrano[ 3',4':6,7]indolizino[1,2-b]quinolin-1-yl]amino}-2-oxoethox y)methylamino glycine
GGFG

(19) United States
(12) Patent Application Publication
(10) Pub. No.: US 2025/0360222 A1
(43) Pub. Date: Nov. 27, 2025

(54) COMBINATION OF ANTIBODY-DRUG CONJUGATE AND BISPECIFIC CHECKPOINT INHIBITOR

(71) Applicants: AstraZeneca UK Limited, Cambridge (GB); DAIICHI SANKYO COMPANY, LIMITED, Tokyo (JP)

(72) Inventors: Jerome Thomas METTETAL, II, Wilmington (DE); Matthew Simon SUNG, Wilmington (DE); Theresa Angela PROIA, Wilmington (DE); Liam Samuel JENKINS, Cambridge (GB)

(73) Assignees: AstraZeneca UK Limited, Cambridge (GB); DAIICHI SANKYO COMPANY, LIMITED, Tokyo (JP)

(21) Appl. No.: 18/997,624

(22) PCT Filed: Jul. 27, 2023
(86) PCT No.: PCT/IB2023/057619

§ 371 Date: Jan. 22, 2025

Related U.S. Application Data
(60) Provisional application No. 63/393,125, filed on Jul. 28, 2022.

(51) Int. Cl. A61K 47/68; A61K 39/00; A61P 35/00; C07K 16/28; C07K 16/30

(52) U.S. Cl. CPC A61K 47/68037; A61K 47/6851; A61K 47/6855; A61P 35/00; C07K 16/2818; C07K 16/30; A61K 2039/507; C07K 2317/31; C07K 2317/76

(57) ABSTRACT

A pharmaceutical product for administration of an antibody-drug conjugate in combination with a bispecific checkpoint inhibitor is provided. The antibody-drug conjugate is an antibody-drug conjugate in which a drug-linker represented by the following formula (wherein A represents the connecting position to an antibody) is conjugated to an antibody, preferably an anti-TROP2 or anti-HER2 antibody, via a thioether bond. Also provided is a therapeutic use and method wherein the antibody-drug conjugate and the bispecific checkpoint inhibitor are administered in combination to a subject:

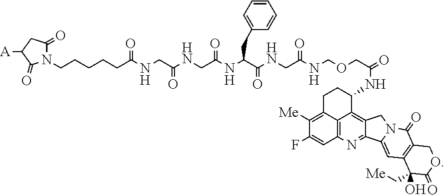

### TECHNICAL FIELD

The present disclosure relates to a pharmaceutical product for administration of a specific antibody-drug conjugate, having an antitumor drug conjugated to an antibody, preferably an anti-TROP2 or anti-HER2 antibody, via a linker structure, in combination with a bispecific checkpoint inhibitor, and to a therapeutic use and method wherein the specific antibody-drug conjugate and the bispecific checkpoint inhibitor are administered in combination to a subject.

### BACKGROUND

Immune checkpoint inhibitors are agents that inhibit the immune suppression system and activate anti-tumor immunity (Menon S. et al., Cancers (2016) 8, 106; Pardoll DM., Nat Rev Cancer (2012) 12, 252-264; Wolchok JD., Cell (2015) 162, 937). Well-validated targets for immune mediated therapy in oncology include PD-1 (programmed cell death protein-1) and CTLA-4 (cytotoxic T-lymphocyte-associated protein 4). TIGIT (T cell immunoreceptor with Ig and ITIM domains), an immune receptor present on some T cells and natural killer cells (NKs), is another target. Examples of known immune checkpoint inhibitors include anti-PD-1 antibodies nivolumab (WO 2006/121168) and pembrolizumab (WO 2008/156712); anti-PD-L1 antibodies atezolizumab (WO 2010/077634), durvalumab (WO 2011/066389), and avelumab (WO 2013/079174); and anti-CTLA-4 antibodies ipilimumab (WO 2001/014424) and tremelimumab (WO 2000/037504).

Bispecific binding proteins that bind specifically to two immune checkpoint targets are under development. Examples include the PD-1/CTLA-4 bispecific antibodies AK104 (cadonilimab) and MEDI5752 (U.S. Pat. No. 10,457,732), which comprise a first domain that binds specifically to PD-1 and a second domain that binds specifically to CTLA-4.

Antibody-drug conjugates (ADCs), which are composed of a cytotoxic drug conjugated to an antibody, can deliver the drug selectively to and within cancer cells, leading to cancer cell death (Ducry, L., et al., Bioconjugate Chem. (2010) 21, 5-13; Alley, S. C., et al., Current Opinion in Chemical Biology (2010) 14, 529-537; Damle N. K. Expert Opin. Biol. Ther. (2004) 4, 1445-1452; Senter P. D., et al., Nature Biotechnology (2012) 30, 631-637; Burris HA., et al., J. Clin. Oncol. (2011) 29(4): 398-405).

One such antibody-drug conjugate is trastuzumab deruxtecan, which is composed of a HER2-targeting antibody and a derivative of exatecan (Ogitani Y. et al., Clinical Cancer Research (2016) 22(20), 5097-5108; Ogitani Y. et al., Cancer Science (2016) 107, 1039-1046). Trastuzumab deruxtecan (Enhertu®, DS-8201) has shown significant clinical efficacy in HER2-expressing solid tumors, including breast cancer, gastric cancer, colorectal cancer and non-small cell lung cancer. Significantly, DS-8201 has demonstrated promising activity in HER2 low tumors in the above indications.

Another such antibody-drug conjugate is datopotamab deruxtecan (DS-1062), which is composed of a TROP2-targeting antibody and a derivative of exatecan. In particular, WO 2015/098099 and WO 2020/240467 provide detailed descriptions of exemplary TROP2-targeting antibody-drug conjugates, including datopotamab deruxtecan (DS-1062). Datopotamab deruxtecan has shown clinical efficacy in multiple tumor types, including lung cancer and breast cancer.

References disclosing combined administration of an antibody-drug conjugate and an immune checkpoint inhibitor include Muller P. et al., Science Translational Medicine (2015) 7(315), 315ra188) (trastuzumab emtansine (T-DM1) combined with both anti-CTLA-4 and anti-PD-1 antibodies); and WO 2018/110515 (trastuzumab deruxtecan (DS-8201) combined with anti-PD-1, anti-PD-L1, anti-CD4 and anti-CD8 antibodies).

However, there is a need to identify further combination partners for antibody-drug conjugates, including for anti-HER2 antibody-drug conjugates such as DS-8201 and for anti-TROP2 antibody-drug conjugates such as DS-1062, to enhance their therapeutic potential.

Despite the therapeutic potential of antibody-drug conjugates such as DS-8201 and DS-1062 as monotherapy or in combination with a checkpoint inhibitor, and the therapeutic potential of bispecific checkpoint inhibitors, a need remains for improved therapeutic compositions and methods that can enhance efficacy of existing cancer treating agents, increase durability of therapeutic response, improve tolerance to patients, reduce dose-dependent toxicity, and/or provide an alternative treatment of cancers exhibiting resistance or refractoriness to a previous cancer treatment.

### SUMMARY OF DISCLOSURE

An antibody-drug conjugate (for example an anti-TROP2 or anti-HER2 antibody-drug conjugate) used in the present disclosure that includes a derivative of the topoisomerase I inhibitor exatecan as a component, has been confirmed to exhibit an excellent antitumor effect in the treatment of certain cancers such as breast cancer, when administered singly or in combination with a checkpoint inhibitor. Furthermore, a bispecific checkpoint inhibitor has been confirmed to exhibit an antitumor effect in the treatment of certain cancers. However, it is desired to provide a medicine and treatment which can obtain a superior antitumor effect in the treatment of cancers, such as enhanced efficacy, increased durability of therapeutic response and/or reduced dose-dependent toxicity.

The present disclosure provides a pharmaceutical product which can exhibit an excellent antitumor effect in the treatment of cancers, through administration of an antibody-drug conjugate, preferably an anti-TROP2 or anti-HER2 antibody-drug conjugate, in combination with a bispecific checkpoint inhibitor, preferably an anti-PD-1/CTLA-4 or anti-PD-1/TIGIT bispecific binding protein. The present disclosure also provides a therapeutic use and method wherein the antibody-drug conjugate and bispecific checkpoint inhibitor are administered in combination to a subject.

Specifically, the present disclosure relates to the following [1] to [93]:

[1] a pharmaceutical product comprising an antibody-drug conjugate and a bispecific checkpoint inhibitor for administration in combination, wherein the antibody-drug conjugate is an antibody-drug conjugate in which a drug-linker represented by the following formula:

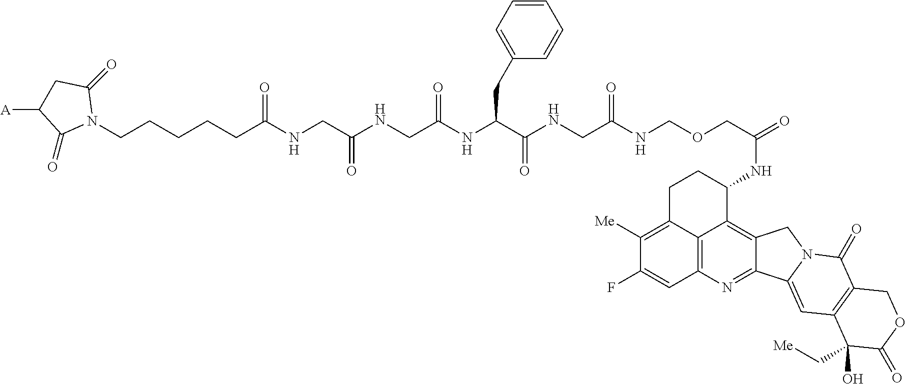

wherein A represents the connecting position to an antibody, is conjugated to an antibody, preferably an anti-TROP2 or anti-HER2 antibody, via a thioether bond;
[2] the pharmaceutical product according to [1], wherein the drug-linker is conjugated to an anti-TROP2 antibody;
[3] the pharmaceutical product according to [2], wherein the anti-TROP2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence represented by SEQ ID NO: 7 and CDRL3 consisting of an amino acid sequence represented by SEQ ID NO: 8;
[4] the pharmaceutical product according to [3], wherein the anti-TROP2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10;
[5] the pharmaceutical product according to [4], wherein the anti-TROP2 antibody is an antibody comprising a heavy chain consisting of an amino acid sequence represented by SEQ ID NO: 12 and a light chain consisting of an amino acid sequence represented by SEQ ID NO: 13;
[6] the pharmaceutical product according to [4], wherein the anti-TROP2 antibody is an antibody comprising a heavy chain consisting of an amino acid sequence represented by SEQ ID NO: 11 and a light chain consisting of an amino acid sequence represented by SEQ ID NO: 13;
[7] the pharmaceutical product according to any one of [2] to [6], wherein the average number of units of the drug-linker conjugated per anti-TROP2 antibody molecule in the antibody-drug conjugate is in the range of from 3.5 to 4.5;
[8] the pharmaceutical product according to [7], wherein the anti-TROP2 antibody-drug conjugate is datopotamab deruxtecan (DS-1062);
[9] the pharmaceutical product according to [1], wherein the drug-linker is conjugated to an anti-HER2 antibody;
[10] the pharmaceutical product according to [9], wherein the anti-HER2 antibody is an antibody comprising a heavy chain comprising CDRH1 consisting of an amino acid sequence represented by SEQ ID NO: 16, CDRH2 consisting of an amino acid sequence represented by SEQ ID NO: 17 and CDRH3 consisting of an amino acid sequence represented by SEQ ID NO: 18, and a light chain comprising CDRL1 consisting of an amino acid sequence represented by SEQ ID NO: 19, CDRL2 consisting of an amino acid sequence consisting of amino acid residues 1 to 3 of SEQ ID NO: 20 and CDRL3 consisting of an amino acid sequence represented by SEQ ID NO: 21;
[11] the pharmaceutical product according to [10], wherein the anti-HER2 antibody is an antibody comprising a heavy chain comprising a heavy chain variable region consisting of an amino acid sequence represented by SEQ ID NO: 22 and a light chain comprising a light chain variable region consisting of an amino acid sequence represented by SEQ ID NO: 23;
[12] the pharmaceutical product according to [11], wherein the anti-HER2 antibody is an antibody comprising a heavy chain consisting of an amino acid sequence represented by SEQ ID NO: 14 and a light chain consisting of an amino acid sequence represented by SEQ ID NO: 15;
[13] the pharmaceutical product according to [11], wherein the anti-HER2 antibody is an antibody comprising a heavy chain consisting of an amino acid sequence represented by SEQ ID NO: 24 and a light chain consisting of an amino acid sequence represented by SEQ ID NO: 15;
[14] the pharmaceutical product according to any one of [9] to [13], wherein the average number of units of the drug-linker conjugated per anti-HER2 antibody molecule in the antibody-drug conjugate is in the range of from 7 to 8;
[15] the pharmaceutical product according to [14], wherein the anti-HER2 antibody-drug conjugate is trastuzumab deruxtecan (DS-8201);
[16] the pharmaceutical product according to any one of [1] to [15], wherein the bispecific checkpoint inhibitor is a bispecific binding protein that comprises a first binding domain that specifically binds to PD-1, and a second binding domain that specifically binds to CTLA-4 or TIGIT;
[17] the pharmaceutical product according to [16], wherein the bispecific binding protein comprises:
a) a first binding domain that specifically binds to PD-1, wherein the first binding domain comprises a heavy chain variable domain comprising a CDRH1 having the amino acid sequence of SEQ ID NO: 25, a CDRH2 having the amino acid sequence of SEQ ID NO: 26, and a CDRH3 having the amino acid sequence of SEQ ID NO: 27, and a light chain variable domain comprising a CDRL1 having the amino acid sequence of SEQ ID NO: 28, a CDRL2 having the amino acid sequence of SEQ ID NO: 29 and a CDRL3 having the amino acid sequence of SEQ ID NO: 30; and
b) a second binding domain that specifically binds to TIGIT, wherein the second binding domain comprises a heavy chain variable domain comprising a CDRH1 having the amino acid sequence of SEQ ID NO: 35, a CDRH2 having the amino acid sequence of SEQ ID NO: 36, and a CDRH3 having the amino acid sequence of SEQ ID NO: 37, and a light chain variable domain comprising a CDRL1 having the amino acid sequence of SEQ ID NO: 38, a CDRL2 having the amino acid sequence of SEQ ID NO: 39, and a CDRL3 having the amino acid sequence of SEQ ID NO: 40;
   [18] the pharmaceutical product according to [17], wherein the first binding domain that specifically binds to PD-1 comprises a heavy chain variable domain having the amino acid sequence of SEQ ID NO: 31 and a light chain variable domain having the amino acid sequence of SEQ ID NO: 33;
   [19] the pharmaceutical product according to [17], wherein first binding domain that specifically binds to PD-1 comprises a heavy chain variable domain having an amino acid sequence that is at least 90% identical to the amino acid sequence of SEQ ID NO: 31 and a light chain variable domain having an amino acid sequence that is at least 90% identical to the amino acid sequence of SEQ ID NO: 33;
   [20] the pharmaceutical product according to any one of [17] to [19], wherein the first binding domain that specifically binds to PD-1 comprises a heavy chain having the amino acid sequence of SEQ ID NO: 32 and a light chain having the amino acid sequence of SEQ ID NO: 34;
   [21] the pharmaceutical product according to any one of [17 to [19], wherein first binding domain that specifically binds to PD-1 comprises a heavy chain having an amino acid sequence that is at least 90% identical to the amino acid sequence of SEQ ID NO: 32 and a light chain having an amino acid sequence that is at least 90% identical to the amino acid sequence of SEQ ID NO: 34;
   [22] the pharmaceutical product according to any one of [17 to [21], wherein the second binding domain that specifically binds TIGIT comprises a heavy chain variable domain having the amino acid sequence of SEQ ID NO: 41 and a light chain variable domain having the amino acid sequence of SEQ ID NO: 43;
   [23] the pharmaceutical product according to any one of [17 to [21], wherein the second binding domain that specifically binds to TIGIT comprises a heavy chain variable domain having an amino acid sequence that is at least 90% identical to the amino acid sequence of SEQ ID NO: 41 and a light chain variable domain having an amino acid sequence that is at least 90% identical to the amino acid sequence of SEQ ID NO: 43;
   [24] the pharmaceutical product according to any one of [17] to [23], wherein the second binding domain that specifically binds to TIGIT comprises a heavy chain having the amino sequence of SEQ ID NO: 42 and a light chain having the amino acid sequence of SEQ ID NO: 44;
   [25] the pharmaceutical product according to any one of [17] to [23], wherein the second binding domain that specifically binds to TIGIT comprises a heavy chain having an amino acid sequence that is at least 90% identical to the amino acid sequence of SEQ ID NO: 42 and a light chain having an amino acid sequence that is at least 90% identical to the amino acid sequence of SEQ ID NO: 44;
   [26] the pharmaceutical product according to [16], wherein the bispecific binding protein comprises:
a) a first binding domain that specifically binds to PD-1, wherein the first binding domain comprises a heavy chain variable domain comprising a CDRH1 having the amino acid sequence of SEQ ID NO: 52, a CDRH2 having the amino acid sequence of SEQ ID NO: 53, and a CDRH3 having the amino acid sequence of SEQ ID NO: 54, and a light chain variable domain comprising a CDRL1 having the amino acid sequence of SEQ ID NO: 49, a CDRL2 having the amino acid sequence of SEQ ID NO: 50 and a CDRL3 having the amino acid sequence of SEQ ID NO: 51; and
b) a second binding domain that specifically binds to CTLA-4, wherein the second binding domain comprises a heavy chain variable domain comprising a CDRH1 having the amino acid sequence of SEQ ID NO: 58, a CDRH2 having the amino acid sequence of SEQ ID NO: 59, and a CDRH3 having the amino acid sequence of SEQ ID NO: 60, and a light chain variable domain comprising a CDRL1 having the amino acid sequence of SEQ ID NO: 55, a CDRL2 having the amino acid sequence of SEQ ID NO: 56, and a CDRL3 having the amino acid sequence of SEQ ID NO: 57;
   [27] the pharmaceutical product according to [26], wherein the first binding domain that specifically binds to PD-1 comprises a heavy chain having the amino acid sequence of SEQ ID NO: 46 and a light chain having the amino acid sequence of SEQ ID NO: 45;
   [28] the pharmaceutical product according to [26], wherein first binding domain that specifically binds to PD-1 comprises a heavy chain having an amino acid sequence that is at least 90% identical to the amino acid sequence of SEQ ID NO: 46 and a light chain having an amino acid sequence that is at least 90% identical to the amino acid sequence of SEQ ID NO: 45;
   [29] the pharmaceutical product according to any one of [26] to [28], wherein the second binding domain that specifically binds to CTLA-4 comprises a heavy chain having the amino sequence of SEQ ID NO: 48 and a light chain having the amino acid sequence of SEQ ID NO: 47;
   [30] the pharmaceutical product according to any one of [26] to [28], wherein the second binding domain that specifically binds to CTLA-4 comprises a heavy chain having an amino acid sequence that is at least 90% identical to the amino acid sequence of SEQ ID NO: 48 and a light chain having an amino acid sequence that is at least 90% identical to the amino acid sequence of SEQ ID NO: 47;
   [31] the pharmaceutical product according to [20] or [27], wherein the light chain constant region is a kappa chain;
   [32] the pharmaceutical product according to [24] or [29], wherein the light chain constant region is a lambda chain;
   [33] the pharmaceutical product according to any one of [16] to [32], wherein the binding protein is an antibody;
   [34] the pharmaceutical product according to [33], wherein the antibody is an IgG antibody;
   [35] the pharmaceutical product according to [34], wherein the antibody is an IgG1 antibody;
   [36] the pharmaceutical product according to [34] or [35], wherein the antibody is human or humanized;
   [37] The pharmaceutical product according to any one of [33] to [36], wherein the bispecific antibody is monovalent;
   [38] the pharmaceutical product according to any one of [16] to [37], wherein the bispecific binding protein is a DuetMab;
   [39] the pharmaceutical product according to any one of [16] to [38], wherein the bispecific binding protein comprises a variant Fc region;
   [40] the pharmaceutical product according to [39], wherein the variant Fc region comprises at least one substitution selected from 221K, 221Y, 225E, 225K, 225W, 228P, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 2621, 262A, 262T, 262E, 2631, 263A, 263T, 263M, 264L, 2641, 264W, 264T, 264R, 264F, 264M, 264Y, 264E, 265A, 265G, 265N, 265Q, 265Y, 265F, 265V, 2651, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 296G, 297S, 297D, 297E, 298A, 298H, 298I, 298T, 298F, 299I, 299L, 299A, 299S, 299V, 299H, 299F, 299E, 305I, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat;
   [41] the pharmaceutical product according to [39], wherein the variant Fc region comprises one or more amino acid substitutions at positions selected from 428 and 434 as numbered by the EU index as set forth in Kabat;
   [42] the pharmaceutical product according to any one of [39] to [41], wherein the variant Fc region comprises one or more amino acid substitutions selected from 428L, 428F, 434A, 434W, and 434Y;
   [43] the pharmaceutical product according to any one of [39] to [42], wherein the variant Fc region comprises a L234F/L235E/P331S triple mutation (TM);
   [44] the pharmaceutical product according to any one of [16] to [43], wherein the bispecific binding protein comprises an Fc region that is aglycosylated;
   [45] the pharmaceutical product according to any one of [16] to [43], wherein the bispecific binding protein comprises an Fc region that is deglycosylated;
   [46] the pharmaceutical product according to any one of [16] to [43], wherein the bispecific binding protein comprises an Fc region that has reduced fucosylation or is afucosylated;
   [47] the pharmaceutical product according to any one of [1] to [46] wherein the product is a combined preparation comprising the antibody-drug conjugate and the bispecific checkpoint inhibitor, for separate simultaneous administration;
   [48] the pharmaceutical product according to any one of [1] to [46] wherein the product is a combined preparation comprising the antibody-drug conjugate and the bispecific checkpoint inhibitor, for sequential or separate simultaneous administration;
   [49] the pharmaceutical product according to any one of [1] to [48], wherein the product is for treating cancer;
   [50] the pharmaceutical product according to [49], wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, melanoma, cervical cancer, uterine cancer, testicular cancer, and renal cell carcinoma;
   [51] the pharmaceutical product according to [50], wherein the cancer is breast cancer;
   [52] the pharmaceutical product according to [51], wherein the breast cancer is HER2 positive breast cancer;
   [53] the pharmaceutical product according to [51], wherein the breast cancer is HER2 low breast cancer;
   [54] the pharmaceutical product according to [51], wherein the breast cancer is triple negative breast cancer;
   [55] the pharmaceutical product according to [51], wherein the breast cancer is hormone receptor (HR)-positive, HER2-negative breast cancer;
   [56] the pharmaceutical product according to [50], wherein the cancer is lung cancer;
   [57] the pharmaceutical product according to [56], wherein the lung cancer is non-small cell lung cancer;
   [58] the pharmaceutical product according to [57], wherein the non-small cell lung cancer is non-small cell lung cancer with actionable genomic alterations;
   [59] the pharmaceutical product according to [57], wherein the non-small cell lung cancer is non-small cell lung cancer lung cancer without actionable genomic alterations;
   [60] the pharmaceutical product according to [50], wherein the cancer is colorectal cancer;
   [61] the pharmaceutical product according to [50], wherein the cancer is gastric cancer;
   [62] the pharmaceutical product according to [50], wherein the cancer is pancreatic cancer;
   [63] the pharmaceutical product according to [50], wherein the cancer is ovarian cancer;
   [64] the pharmaceutical product according to [50], wherein the cancer is prostate cancer;
   [65] the pharmaceutical product according to [50], wherein the cancer is kidney cancer;
   [66] the pharmaceutical product according to [50], wherein the cancer is bladder cancer;
   [67] the pharmaceutical product according to [50], wherein the cancer is endometrial cancer;
   [68] the pharmaceutical product according to [50], wherein the cancer is biliary tract cancer;
   [69] a pharmaceutical product as defined in any one of [1] to [48], for use in treating cancer;
   [70] the pharmaceutical product for the use according to [69], wherein the cancer is as defined in any one of [50] to [68];
   [71] the pharmaceutical product according to any one of [1] to [48], further comprising carboplatin for administration in combination with the antibody-drug conjugate and the bispecific checkpoint inhibitor;
   [72] the pharmaceutical product according to any one of [1] to [48], further comprising a fluoropyrimidine for administration in combination with the antibody-drug conjugate and the bispecific checkpoint inhibitor;
   [73] Use of an antibody-drug conjugate in the manufacture of a medicament for use in combination with a bispecific checkpoint inhibitor, wherein the antibody-drug conjugate and the bispecific checkpoint inhibitor are as defined in any one of [1] to [46], for treating cancer;
   [74] the use according to [73] wherein the medicament is for use in combination with the bispecific checkpoint inhibitor by sequential administration;
   [75] the use according to [73] wherein the medicament is for use in combination with the bispecific checkpoint inhibitor by separate simultaneous administration;
   [76] Use of a bispecific checkpoint inhibitor in the manufacture of a medicament for use in combination with an antibody-drug conjugate, wherein the antibody-drug conjugate and the bispecific checkpoint inhibitor are as defined in any one of [1] to [46], for treating cancer;
   [77] the use according to [76] wherein the medicament is for use in combination with the antibody-drug conjugate by sequential administration;
   [78] the use according to [76] wherein the medicament is for use in combination with the antibody-drug conjugate by separate simultaneous administration;
   [79] the use according to any one of [73] to [78], wherein the cancer is as defined in any one of [50] to [68];
   [80] an antibody-drug conjugate for use, in combination with a bispecific checkpoint inhibitor, in the treatment of cancer, wherein the antibody-drug conjugate and the bispecific checkpoint inhibitor are as defined in any one of [1] to [46];
   [81] the antibody-drug conjugate for the use according to [80], wherein the cancer is as defined in any one of [50] to [68];
   [82] the antibody-drug conjugate for the use according to [80] or [81], wherein the use comprises administration of the antibody-drug conjugate and the bispecific checkpoint inhibitor sequentially;
   [83] the antibody-drug conjugate for the use according to [80] or [81], wherein the use comprises administration of the antibody-drug conjugate and the bispecific checkpoint inhibitor separately and simultaneously;
   [84] an antibody-drug conjugate for use in the treatment of cancer in a subject, wherein said treatment comprises the sequential or separate simultaneous administration of i) the antibody-drug conjugate, and ii) a bispecific checkpoint inhibitor to said subject, wherein the antibody-drug conjugate and the bispecific checkpoint inhibitor are as defined in any one of [1] to [46];
   [85] a bispecific checkpoint inhibitor for use, in combination with an antibody-drug conjugate, in the treatment of cancer, wherein the antibody-drug conjugate and the bispecific checkpoint inhibitor are as defined in any one of [1] to [46];
   [86] the bispecific checkpoint inhibitor for the use according to [85], wherein the cancer is as defined in any one of [50] to [68];
   [87] the bispecific checkpoint inhibitor for the use according to [85] or [86], wherein the use comprises administration of the antibody-drug conjugate and the bispecific checkpoint inhibitor sequentially;
   [88] the bispecific checkpoint inhibitor for the use according to [85] or [86], wherein the use comprises administration of the antibody-drug conjugate and the bispecific checkpoint inhibitor separately and simultaneously;
   [89] a bispecific checkpoint inhibitor for use in the treatment of cancer in a subject, wherein said treatment comprises the sequential or separate simultaneous administration of i) the bispecific checkpoint inhibitor, and ii) an antibody-drug conjugate to said subject, wherein the bispecific checkpoint inhibitor and the antibody-drug conjugate are as defined in any one of [1] to [46];
   [90] a method of treating cancer comprising administering an antibody-drug conjugate and a bispecific checkpoint inhibitor as defined in any one of [1] to [46] in combination to a subject in need thereof;
   [91] the method according to [90], wherein the cancer is as defined in any one of [50] to [68];
   [92] the method according to [90] or [91], wherein the method comprises administering the antibody-drug conjugate and the bispecific checkpoint inhibitor sequentially; and
   [93] the method according to [90] or [91], wherein the method comprises administering the antibody-drug conjugate and the bispecific checkpoint inhibitor separately and simultaneously.

### Advantageous Effects of Disclosure

The present disclosure provides a pharmaceutical product comprising a specified antibody-drug conjugate, having an antitumor drug conjugated to an antibody (preferably an anti-TROP2 or anti-HER2 antibody) via a linker structure, and a bispecific checkpoint inhibitor, for administration in combination, and a therapeutic use and method wherein the specified antibody-drug conjugate and the bispecific checkpoint inhibitor are administered in combination to a subject. Thus, the present disclosure provide a medicine and treatment which can obtain a superior antitumor effect in the treatment of cancers.

### BRIEF DESCRIPTION OF DRAWINGS

### [Anti-TROP2 Antibody]:

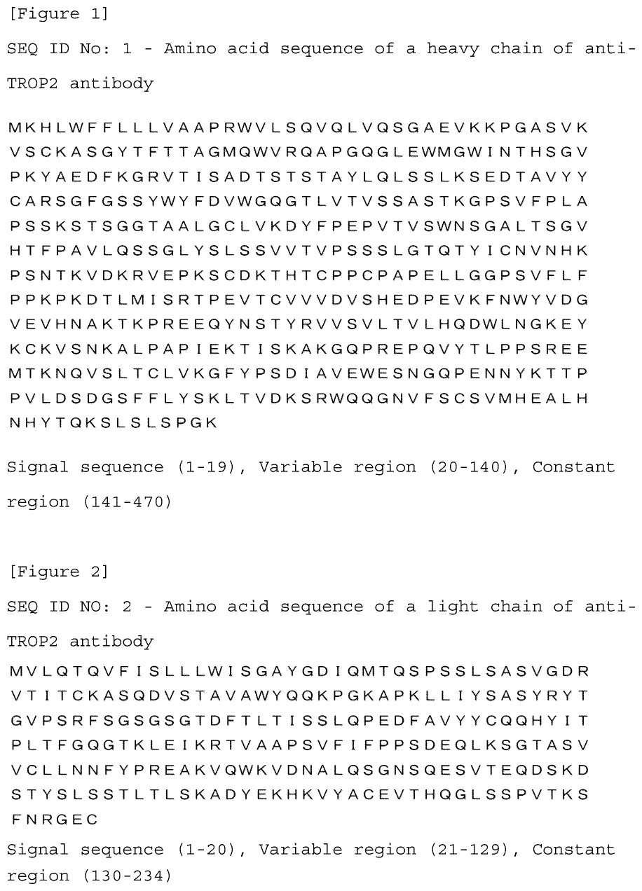

FIG. 1 is a diagram showing the amino acid sequence of a heavy chain of an anti-TROP2 antibody (SEQ ID NO: 1).

FIG. 2 is a diagram showing the amino acid sequence of a light chain of an anti-TROP2 antibody (SEQ ID NO: 2).

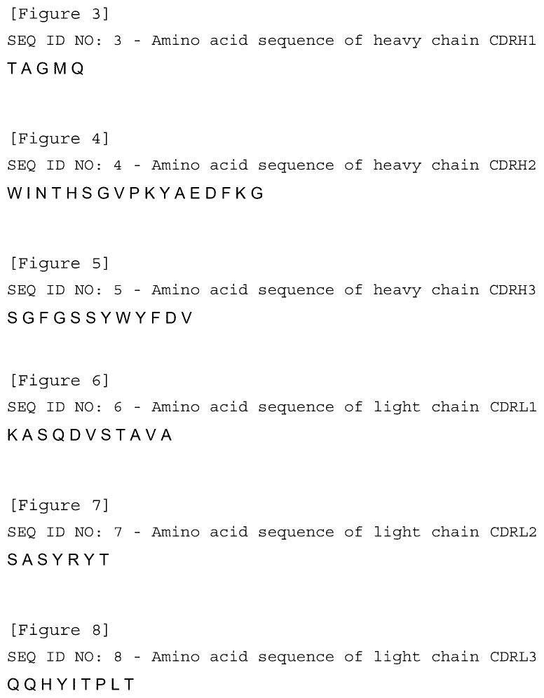

FIG. 3 is a diagram showing the amino acid sequence of a heavy chain CDRH1 (SEQ ID NO: 3 [=amino acid residues 50 to 54 of SEQ ID NO: 1]).

FIG. 4 is a diagram showing the amino acid sequence of a heavy chain CDRH2 (SEQ ID NO: 4 [=amino acid residues 69 to 85 of SEQ ID NO: 1]).

FIG. 5 is a diagram showing the amino acid sequence of a heavy chain CDRH3 (SEQ ID NO: 5 [=amino acid residues 118 to 129 of SEQ ID NO: 1]).

FIG. 6 is a diagram showing the amino acid sequence of a light chain CDRL1 (SEQ ID NO: 6 [=amino acid residues 44 to 54 of SEQ ID NO: 2]).

FIG. 7 is a diagram showing the amino acid sequence of a light chain CDRL2 (SEQ ID NO: 7 [=amino acid residues 70 to 76 of SEQ ID NO: 2]).

FIG. 8 is a diagram showing the amino acid sequence of a light chain CDRL3 (SEQ ID NO: 8 [=amino acid residues 109 to 117 of SEQ ID NO: 2]).

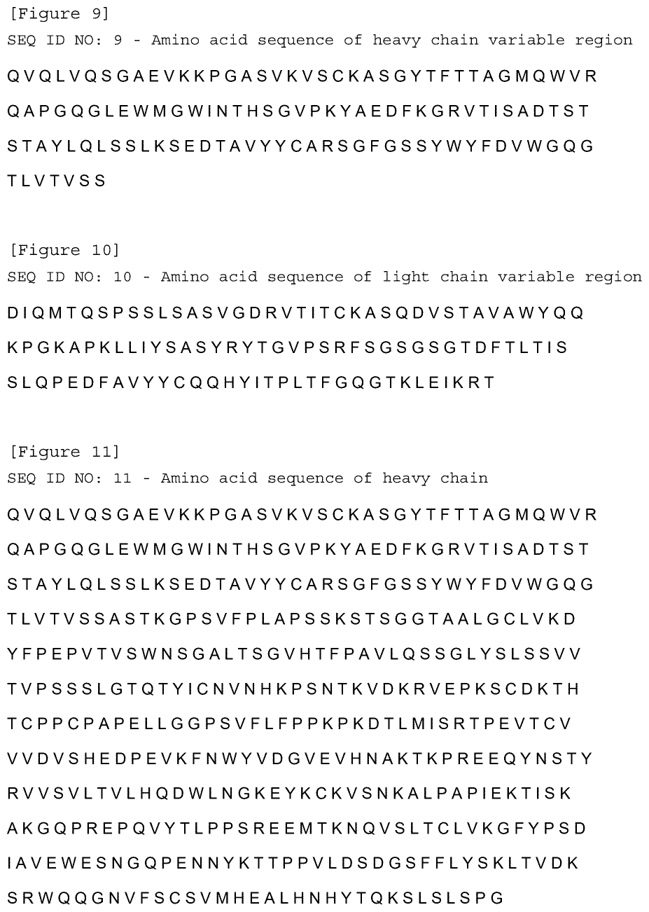

FIG. 9 is a diagram showing the amino acid sequence of a heavy chain variable region (SEQ ID NO: 9 [=amino acid residues 20 to 140 of SEQ ID NO: 1]).

FIG. 10 is a diagram showing the amino acid sequence of a light chain variable region (SEQ ID NO: 10 [=amino acid residues 21 to 129 of SEQ ID NO: 2]).

FIG. 11 is a diagram showing the amino acid sequence of a heavy chain (SEQ ID NO: 11 [=amino acid residues 20 to 469 of SEQ ID NO: 1]).

### [Anti-HER2 Antibody]:

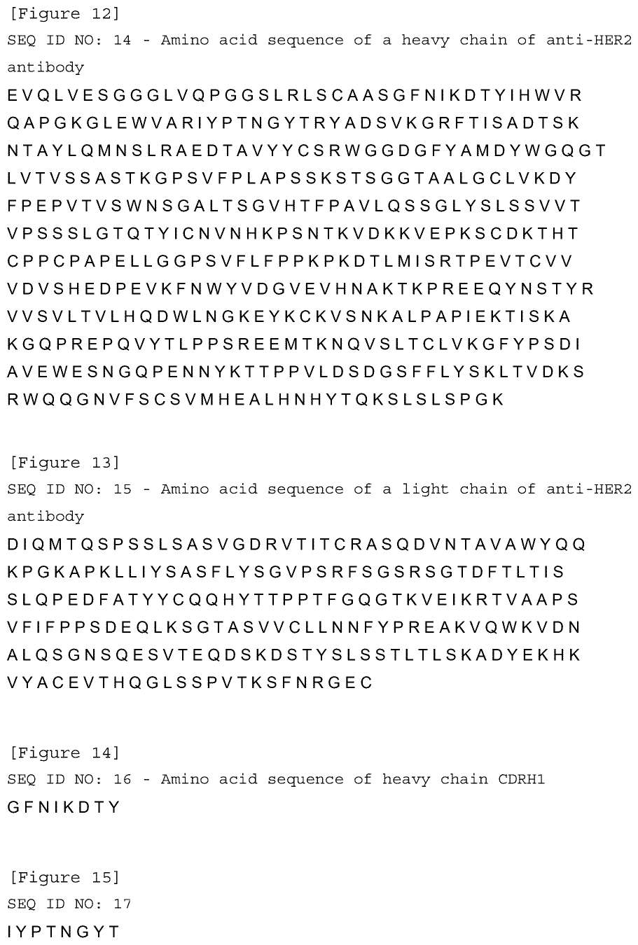

FIG. 12 is a diagram showing the amino acid sequence of a heavy chain of an anti-HER2 antibody (SEQ ID NO: 14).

FIG. 13 is a diagram showing the amino acid sequence of a light chain of an anti-HER2 antibody (SEQ ID NO: 15).

FIG. 14 is a diagram showing the amino acid sequence of a heavy chain CDRH1 (SEQ ID NO: 16 [=amino acid residues 26 to 33 of SEQ ID NO: 14]).

FIG. 15 is a diagram showing the amino acid sequence of a heavy chain CDRH2 (SEQ ID NO: 17 [=amino acid residues 51 to 58 of SEQ ID NO: 14]).

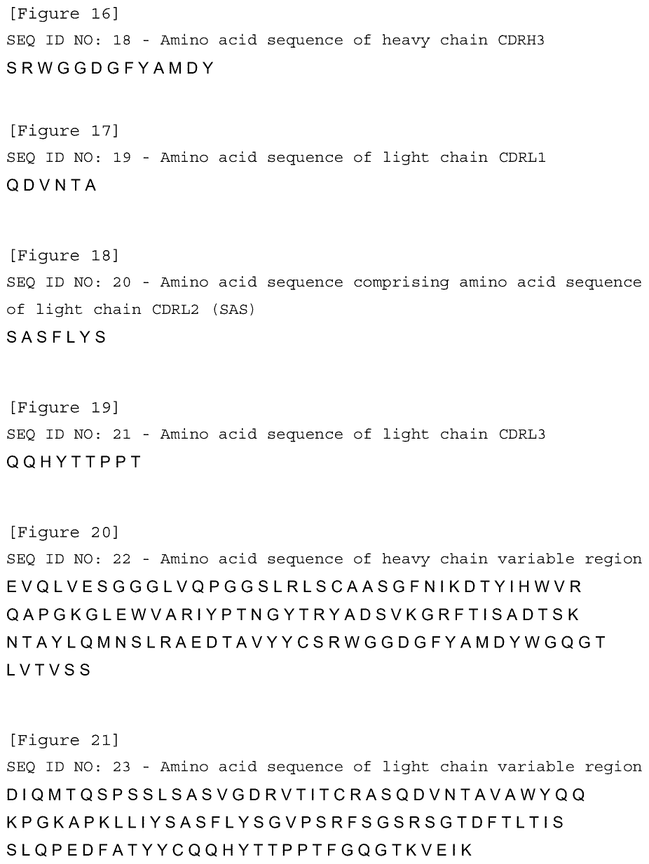

FIG. 16 is a diagram showing the amino acid sequence of a heavy chain CDRH3 (SEQ ID NO: 18 [=amino acid residues 97 to 109 of SEQ ID NO: 14]).

FIG. 17 is a diagram showing the amino acid sequence of a light chain CDRL1 (SEQ ID NO: 19 [=amino acid residues 27 to 32 of SEQ ID NO: 15]).

FIG. 18 is a diagram showing an amino acid sequence comprising the amino acid sequence of a light chain CDRL2 (SAS) (SEQ ID NO: 20 [=amino acid residues 50 to 56 of SEQ ID NO: 15]).

FIG. 19 is a diagram showing the amino acid sequence of a light chain CDRL3 (SEQ ID NO: 21 [=amino acid residues 89 to 97 of SEQ ID NO: 15]).

FIG. 20 is a diagram showing the amino acid sequence of a heavy chain variable region (SEQ ID NO: 22 [=amino acid residues 1 to 120 of SEQ ID NO: 14]).

FIG. 21 is a diagram showing the amino acid sequence of a light chain variable region (SEQ ID NO: 23 [=amino acid residues 1 to 107 of SEQ ID NO: 15]).

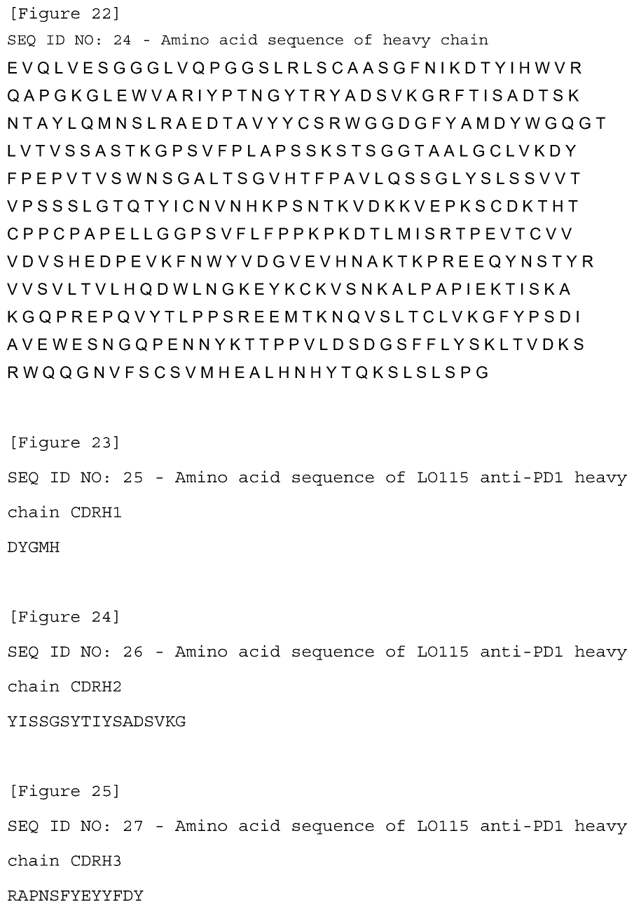

FIG. 22 is a diagram showing the amino acid sequence of a heavy chain (SEQ ID NO: 24 [=amino acid residues 1 to 449 of SEQ ID NO: 14]).

### [Anti-PD-1/TIGIT Bispecific Antibody]:

FIG. 23 is a diagram showing the amino acid sequence of an anti-PD1 heavy chain CDRH1 (SEQ ID NO: 25)

FIG. 24 is a diagram showing the amino acid sequence of an anti-PD1 heavy chain CDRH2 (SEQ ID NO: 26)

FIG. 25 is a diagram showing the amino acid sequence of an anti-PD1 heavy chain CDRH3 (SEQ ID NO: 27)

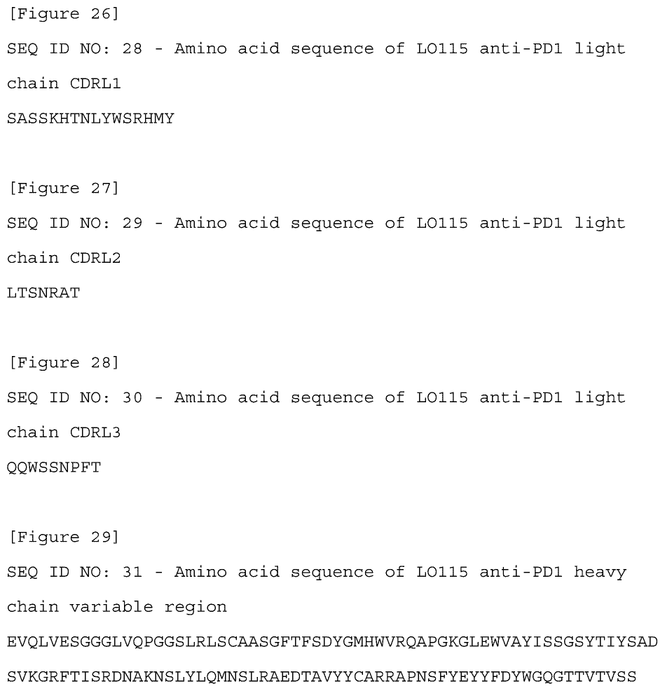

FIG. 26 is a diagram showing the amino acid sequence of an anti-PD1 light chain CDRL1 (SEQ ID NO: 28)

FIG. 27 is a diagram showing the amino acid sequence of an anti-PD1 light chain CDRL2 (SEQ ID NO: 29)

FIG. 28 is a diagram showing the amino acid sequence of an anti-PD1 light chain CDRL3 (SEQ ID NO: 30)

FIG. 29 is a diagram showing the amino acid sequence of an anti-PD1 heavy chain variable region (SEQ ID NO: 31)

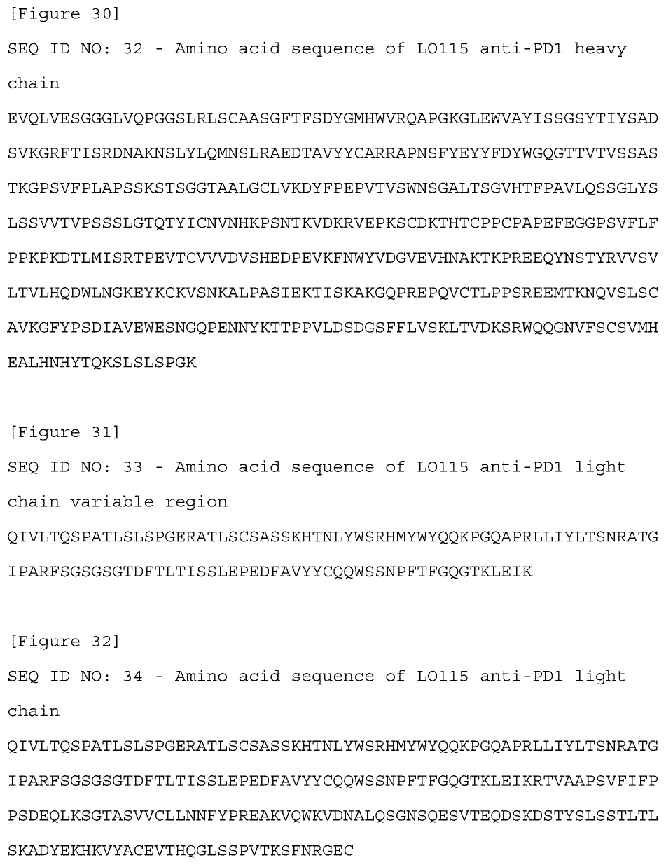

FIG. 30 is a diagram showing the amino acid sequence of an anti-PD1 heavy chain (SEQ ID NO: 32)

FIG. 31 is a diagram showing the amino acid sequence of an anti-PD1 light chain variable region (SEQ ID NO: 33)

FIG. 32 is a diagram showing the amino acid sequence of an anti-PD1 light chain (SEQ ID NO: 34)

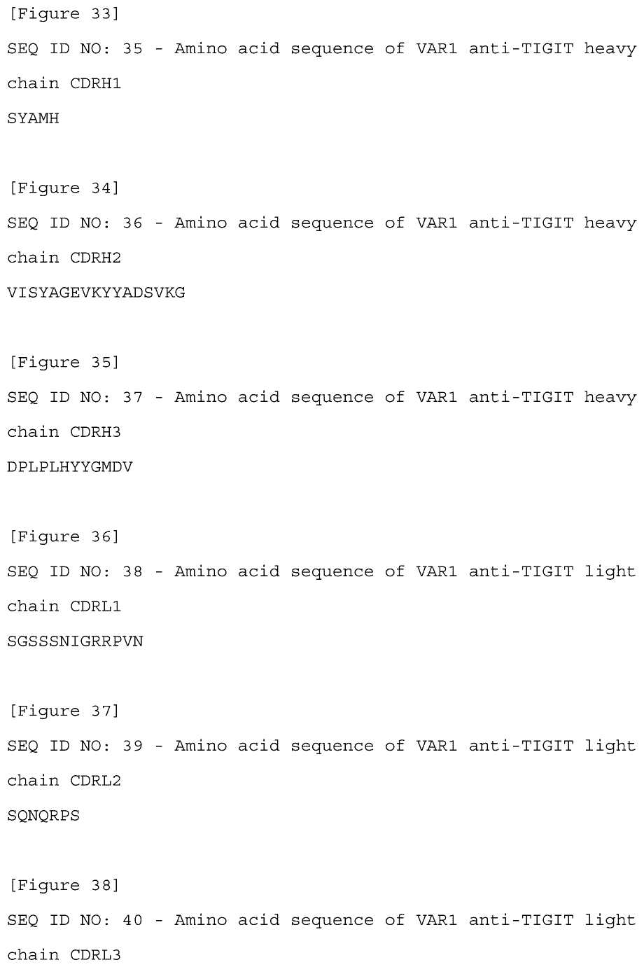

FIG. 33 is a diagram showing the amino acid sequence of an anti-TIGIT heavy chain CDRH1 (SEQ ID NO: 35)

FIG. 34 is a diagram showing the amino acid sequence of an anti-TIGIT heavy chain CDRH2 (SEQ ID NO: 36)

FIG. 35 is a diagram showing the amino acid sequence of an anti-TIGIT heavy chain CDRH3 (SEQ ID NO: 37)

FIG. 36 is a diagram showing the amino acid sequence of an anti-TIGIT light chain CDRL1 (SEQ ID NO: 38)

FIG. 37 is a diagram showing the amino acid sequence of an anti-TIGIT light chain CDRL2 (SEQ ID NO: 39)

FIG. 38 is a diagram showing the amino acid sequence of an anti-TIGIT light chain CDRL3 (SEQ ID NO: 40)

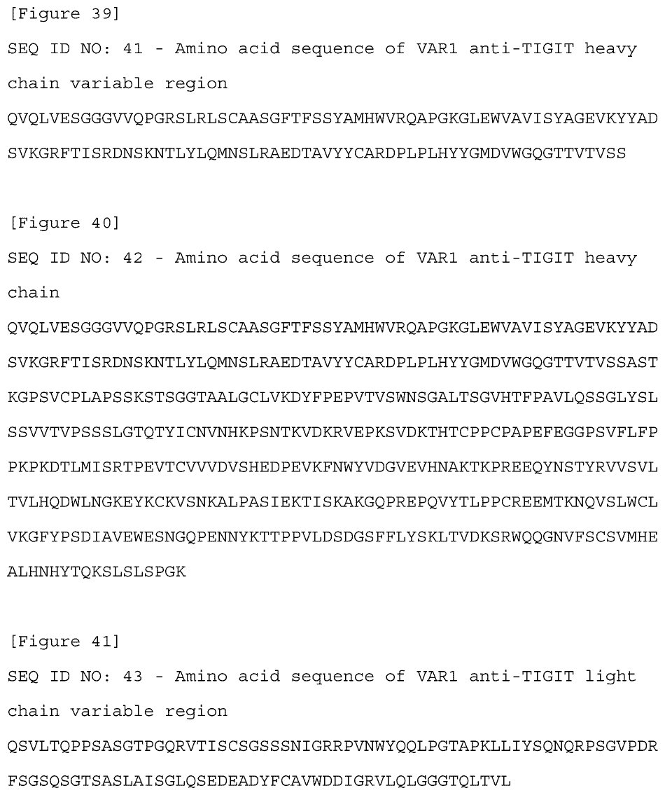

FIG. 39 is a diagram showing the amino acid sequence of an anti-TIGIT heavy chain variable region (SEQ ID NO: 41)

FIG. 40 is a diagram showing the amino acid sequence of an anti-TIGIT heavy chain (SEQ ID NO: 42)

FIG. 41 is a diagram showing the amino acid sequence of an anti-TIGIT light chain variable region (SEQ ID NO: 43)

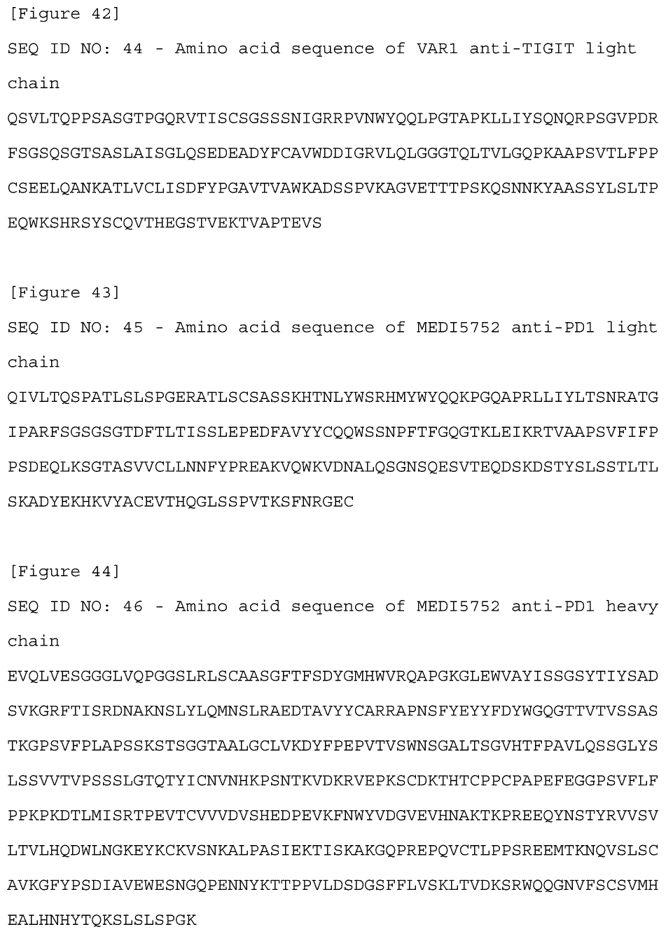

FIG. 42 is a diagram showing the amino acid sequence of an anti-TIGIT light chain (SEQ ID NO: 44) [anti-PD-1/CTLA-4 bispecific antibody]:

FIG. 43 is a diagram showing the amino acid sequence of an anti-PD1 light chain (SEQ ID NO: 45)

FIG. 44 is a diagram showing the amino acid sequence of an anti-PD1 heavy chain (SEQ ID NO: 46)

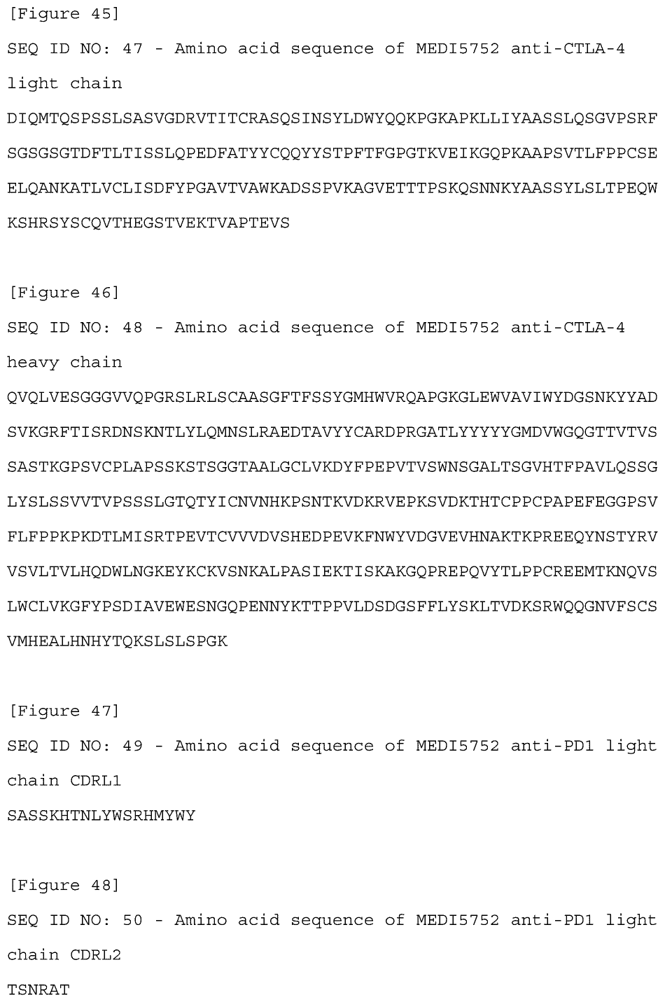

FIG. 45 is a diagram showing the amino acid sequence of an anti-CTLA-4 light chain (SEQ ID NO: 47)

FIG. 46 is a diagram showing the amino acid sequence of an anti-CTLA-4 heavy chain (SEQ ID NO: 48)

FIG. 47 is a diagram showing the amino acid sequence of an anti-PD1 light chain CDRL1 (SEQ ID NO: 49)

FIG. 48 is a diagram showing the amino acid sequence of an anti-PD1 light chain CDRL2 (SEQ ID NO: 50)

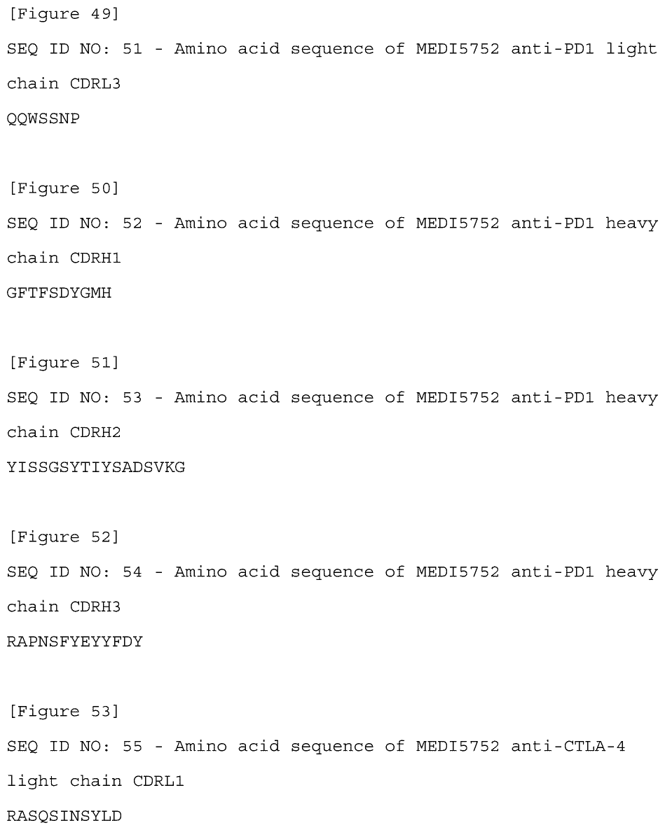

FIG. 49 is a diagram showing the amino acid sequence of an anti-PD1 light chain CDRL3 (SEQ ID NO: 51)

FIG. 50 is a diagram showing the amino acid sequence of an anti-PD1 heavy chain CDRH1 (SEQ ID NO: 52)

FIG. 51 is a diagram showing the amino acid sequence of an anti-PD1 heavy chain CDRH2 (SEQ ID NO: 53)

FIG. 52 is a diagram showing the amino acid sequence of an anti-PD1 heavy chain CDRH3 (SEQ ID NO: 54)

FIG. 53 is a diagram showing the amino acid sequence of an anti-CTLA-4 light chain CDRL1 (SEQ ID NO: 55)

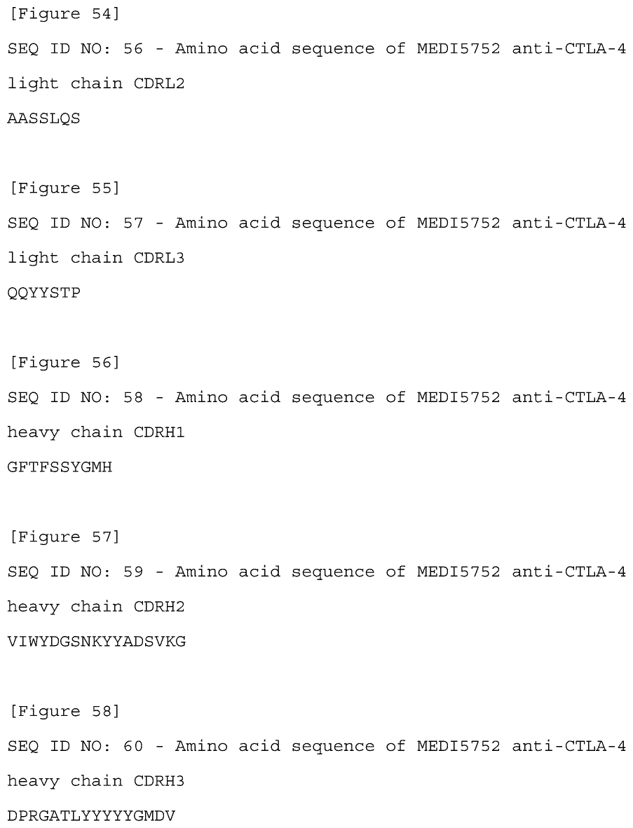

FIG. 54 is a diagram showing the amino acid sequence of an anti-CTLA-4 light chain CDRL2 (SEQ ID NO: 56)

FIG. 55 is a diagram showing the amino acid sequence of an anti-CTLA-4 light chain CDRL3 (SEQ ID NO: 57)

FIG. 56 is a diagram showing the amino acid sequence of an anti-CTLA-4 heavy chain CDRH1 (SEQ ID NO: 58)

FIG. 57 is a diagram showing the amino acid sequence of an anti-CTLA-4 heavy chain CDRH2 (SEQ ID NO: 59)

FIG. 58 is a diagram showing the amino acid sequence of an anti-CTLA-4 heavy chain CDRH3 (SEQ ID NO: 60)

### [Experimental]:

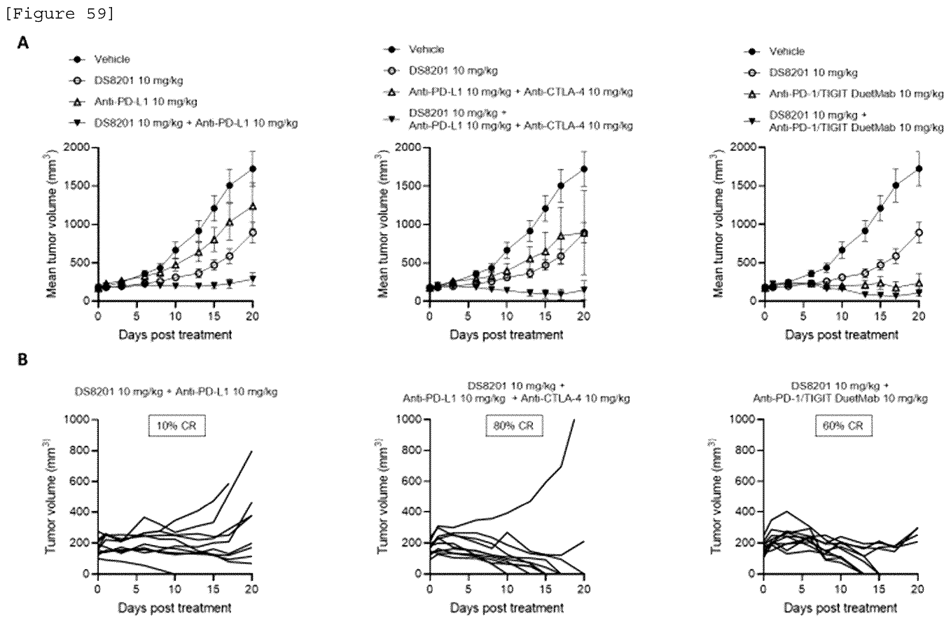

FIG. 59 represents graphs (A) showing mean changes in tumor volume over time, ±SEM (n=10 mice per treatment group), and (B) changes in tumor volume over time for individual mice (CR=complete response), in EMT6 hHER2-tumor bearing BALB/c mice treated with 10 mg/kg DS-8201 alone, or in combination with 10 mg/kg anti-PD-L1, 10 mg/kg anti-PD-L1+10 mg/kg anti-CTLA-4 and 10 mg/kg anti-PD-1/TIGIT DuetMab.

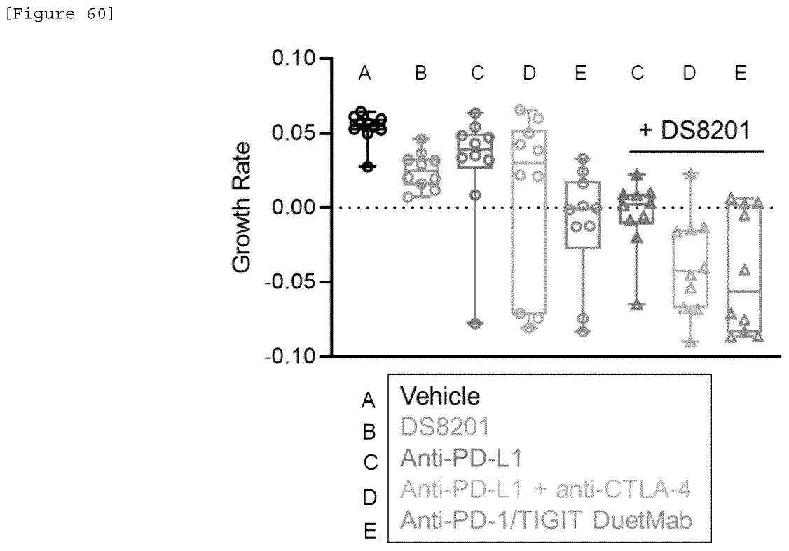

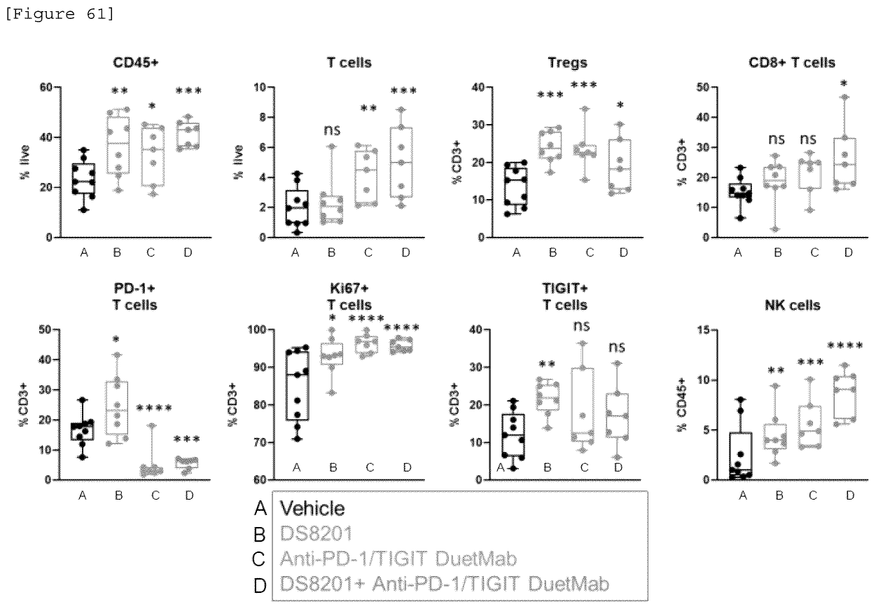

FIG. 60 represents a graph showing tumor growth rates (n=10 mice per treatment group) in EMT6 hHER2-tumor bearing BALB/c mice treated with 10 mg/kg DS-8201 alone, or in combination with 10 mg/kg anti-PD-L1, 10 mg/kg anti-PD-L1+10 mg/kg anti-CTLA-4 and 10 mg/kg anti-PD-1/TIGIT DuetMab.

FIG. 61 represents graphs reporting pharmacodynamic changes assessed via flow cytometry, at 8 days post initiation of treatment in EMT6 hHER2-tumor bearing BALB/c mice treated with treated with 10 mg/kg DS-8201 alone, or in combination with 10 mg/kg anti-PD-1/TIGIT DuetMab.

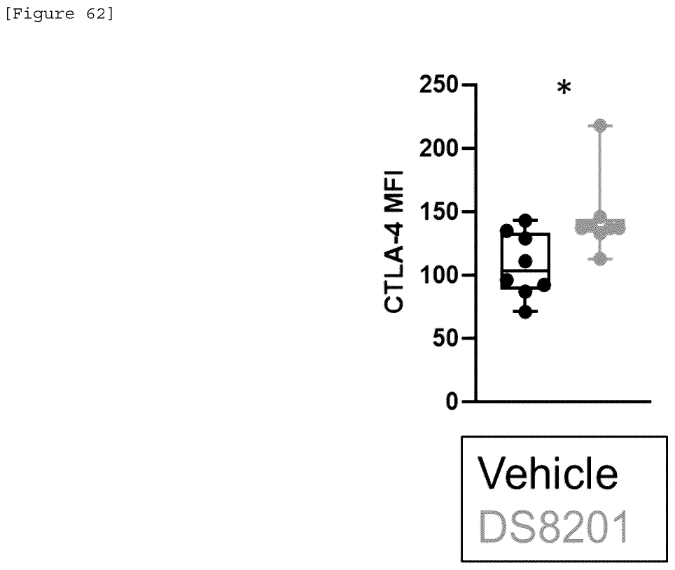

FIG. 62 represents a graph reporting changes in tumoral CD8+ T cell CTLA-4 expression assessed via flow cytometry, at 10 days post initiation of treatment in EMT6 hHER2-bearing BALB/c mice treated with 10 mg/kg DS-8201.

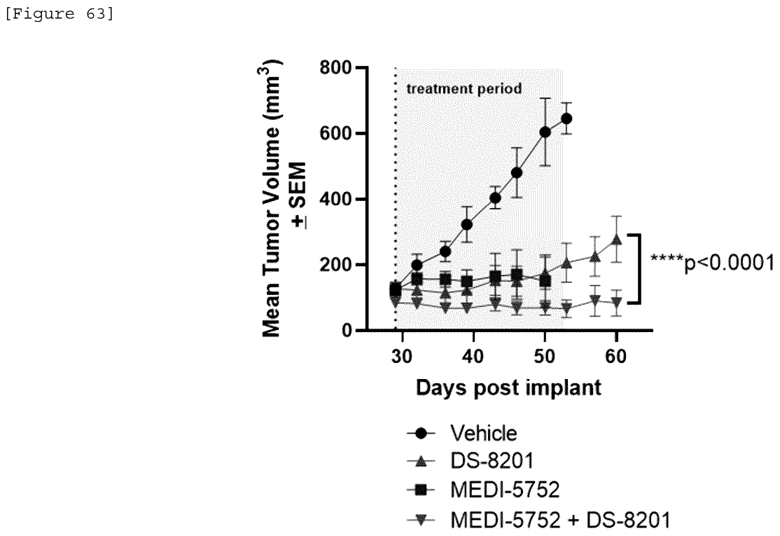

FIG. 63 represents a graph showing tumor growth rates in a CD34+ humanized mouse bearing Caki-1 tumors treated with a single dose of DS-8201 at 10 mg/kg IV alone, MEDI5752 10 mg/kg IP alone biweekly, or the combination of DS-8201 and MEDI5752 each administered at 10 mg/kg.

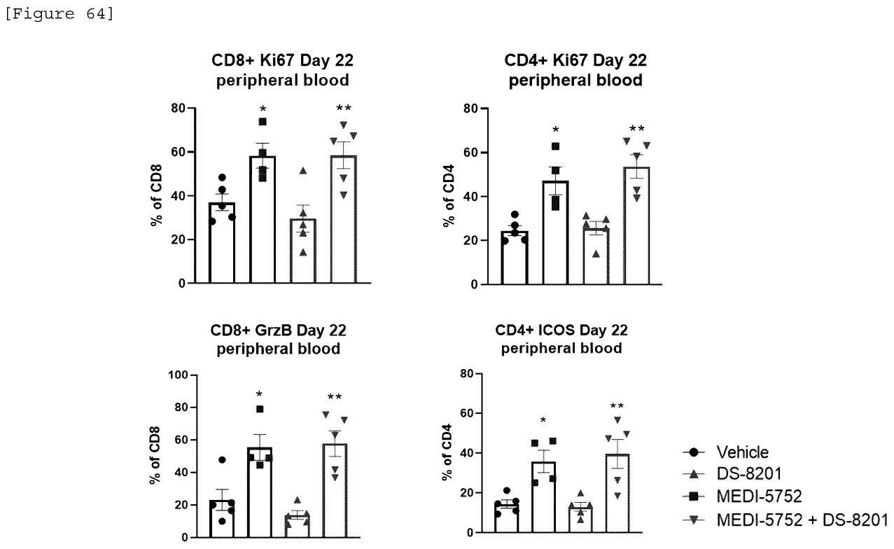

FIG. 64 represents a graph reporting changes in peripheral blood populations of CD8+ and CD4+ T cells assessed via flow cytometry, at 22 days post initiation of treatment with DS-8201, MEDI5752, or the combination of DS-8201 and MEDI5752.

### DETAILED DESCRIPTION

In order that the present disclosure can be more readily understood, certain terms are first defined. Additional definitions are set forth throughout the detailed description.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Unless otherwise required by context, singular terms shall include pluralities and plural terms shall include the singular.

Units, prefixes, and symbols are denoted in their Système International de Unites (SI) accepted form. Numeric ranges are inclusive of the numbers defining the range.

It is understood that wherever aspects are described herein with the language “comprising”, otherwise analogous aspects described in terms of “consisting of” and/or “consisting essentially of” are also provided.

The terms “inhibit” and “inhibition” can refer to a decrease of about 10%, 20%, 30%, 40%, 50%, 60%, 70%, 80%, 90% or 100% in biological activity. Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical product” refers to a preparation which is in such form as to permit the biological activity of the active ingredients, either as a composition containing all the active ingredients (for simultaneous administration), or as a combination of separate compositions (a combined preparation) each containing at least one but not all of the active ingredients (for administration sequentially or simultaneously), and which contains no additional components which are unacceptably toxic to a subject to which the product would be administered. Such product can be sterile. By “simultaneous administration” is meant that the active ingredients are administered at the same time. By “sequential administration” is meant that the active ingredients are administered one after the other, in either order, at a time interval between the individual administrations. The time interval can be, for example, less than 24 hours, preferably less than 6 hours, more preferably less than 2 hours.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods of the present disclosure if the patient shows, e.g., total, partial, or transient remission of a certain type of cancer.

The terms “cancer”, “tumor”, “cancerous”, and “malignant” refer to or describe the physiological condition in mammals that is typically characterized by unregulated cell growth. Examples of cancers include but are not limited to, breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, melanoma, cervical cancer, uterine cancer, testicular cancer, and renal cell carcinoma. Cancers include hematological malignancies such as acute myeloid leukemia, multiple myeloma, chronic lymphocytic leukemia, diffuse large B cell lymphoma, Burkitt's lymphoma, follicular lymphoma and solid tumors such as breast cancer, lung cancer, neuroblastoma and colon cancer.

The term “cytotoxic drug” as used herein is defined broadly and refers to a substance that inhibits or prevents the function of cells and/or causes destruction of cells (cell death), and/or exerts anti-neoplastic/anti-proliferative effects. For example, a cytotoxic drug prevents directly or indirectly the development, maturation, or spread of neoplastic tumor cells. The term includes also such agents that cause a cytostatic effect only and not a mere cytotoxic effect. The term includes chemotherapeutic agents as specified below.

The term “chemotherapeutic agent” is a subset of the term “cytotoxic drug” comprising natural or synthetic chemical compounds.

In accordance with the methods or uses of the present disclosure, compounds of the present disclosure may be administered to a patient to promote a positive therapeutic response with respect to cancer. The term “positive therapeutic response” with respect to cancer treatment refers to an improvement in the symptoms associated with the disease. For example, an improvement in the disease can be characterized as a complete response. The term “complete response” refers to an absence of clinically detectable disease with normalization of any previous test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of compounds of the present disclosure. In specific aspects, such terms refer to one, two or three or more results following the administration of compounds of the instant disclosure:
(1) a stabilization, reduction or elimination of the cancer cell population;
(2) a stabilization or reduction in cancer growth; (3) an impairment in the formation of cancer;
(4) eradication, removal, or control of primary, regional and/or metastatic cancer;
(5) a reduction in mortality;
(6) an increase in disease-free, relapse-free, progression-free, and/or overall survival, duration, or rate;
(7) an increase in the response rate, the durability of response, or number of patients who respond or are in remission;
(8) a decrease in hospitalization rate,
(9) a decrease in hospitalization lengths,
(10) the size of the cancer is maintained and does not increase or increases by less than 10%, preferably less than 5%, preferably less than 4%, preferably less than 2%, and
(11) an increase in the number of patients in remission.
(12) a decrease in the number of adjuvant therapies (e.g., chemotherapy or hormonal therapy) that would otherwise be required to treat the cancer.

Clinical response can be assessed using screening techniques such as PET, magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy can experience the beneficial effect of an improvement in the symptoms associated with the disease.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain”, as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. As used herein, the terms “variable region” or “variable domain” are used interchangeably and are common in the art. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the CH₃ domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus. Those of skill in the art, however, would appreciate that the locations of the domains in a naturally occurring antibody can be modified in certain antibody-like binding protein formats without a loss of antigen-binding capability. Classes of human light chains are termed kappa and lambda light chains.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “antibody fragment” refers to a portion of an intact or full-length chain or an antibody, generally the target binding or variable region. Examples of antibody fragments include, but are not limited to, Fab, F_{ab′}, F_{(ab′)2} and Fᵥ fragments. As used herein, the term “functional fragment” is generally synonymous with “antibody fragment”, and with respect to antibodies, can refer to antibody fragments such as Fᵥ, F_{ab}, F_{(ab′)2}.

Reference to the numbering of amino acid residues described herein is performed according to the EU numbering system (also described in Kabat et al, Sequences of Proteins of Immunological Interest, 5th Ed. Public Health Service, National Institutes of Health, Bethesda, MD. (1991)).

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in manner including but not limited to by hybridoma, phage selection, recombinant expression, and transgenic animals.

The term “human antibody”, as used herein, includes antibodies having variable and constant regions substantially corresponding to human germline immunoglobulin sequences. In some aspects, human antibodies are produced in non-human mammals, including, but not limited to, rodents, such as mice and rats, and lagomorphs, such as rabbits. In other aspects, human antibodies are produced in hybridoma cells. In still other aspects, human antibodies are produced recombinantly. In some aspects, the bispecific binding protein is a human or humanized antibody.

The term “antigen” or “target antigen” as used herein refers to a molecule or a portion of a molecule that is capable of being recognized by and bound by binding proteins of the disclosure. The target antigen is capable of being used in an animal to produce antibodies capable of binding to an epitope of that antigen. A target antigen may have one or more epitopes.

The term “epitope” as used herein refers to a region or structural element of an antigen that is recognized and bound by a binding protein of the disclosure. More precisely, the epitope is the specific structure that is bound by the CDRs of the binding protein. Epitopes can comprise protein structural elements, carbohydrates or even portions of lipid structures found in membranes. A binding protein is said to specifically bind an antigen when it preferentially recognizes its antigen target in a complex mixture of proteins and/or macromolecules. The term “specifically binds” refers to a binding protein that specifically binds to a molecule or a fragment thereof (e.g., antigen). A binding protein that specifically binds a molecule or a fragment thereof may bind to other molecules with lower affinity as determined by, for example, immunoassays, BIAcore, or other assays known in the art. In particular, antibodies or fragments that specifically bind to at least one molecule or a fragment thereof can compete off molecules that bind non-specifically. The present disclosure specifically encompasses antibodies with multiple specificities (e.g., an antibody with specificity for two or more discrete antigens. For example, a bispecific antibody can bind to two adjacent epitopes on a single target antigen, or can bind to two different antigens.

The term “antigen binding site” as used herein refers to a site created on the surface of a binding protein of the disclosure where an antigen or an epitope on an antigen is bound. The antigen binding site of the binding protein is typically described by reference to the loop structures created by complementarity determining regions (CDRs) of the binding protein.

### Description of Embodiments

Hereinafter, preferred modes for carrying out the present disclosure are described. The embodiments described below are given merely for illustrating one example of a typical embodiment of the present disclosure and are not intended to limit the scope of the present disclosure.

### 1. Antibody-Drug Conjugate

The antibody-drug conjugate used in the present disclosure is an antibody-drug conjugate in which a drug-linker represented by the following formula:

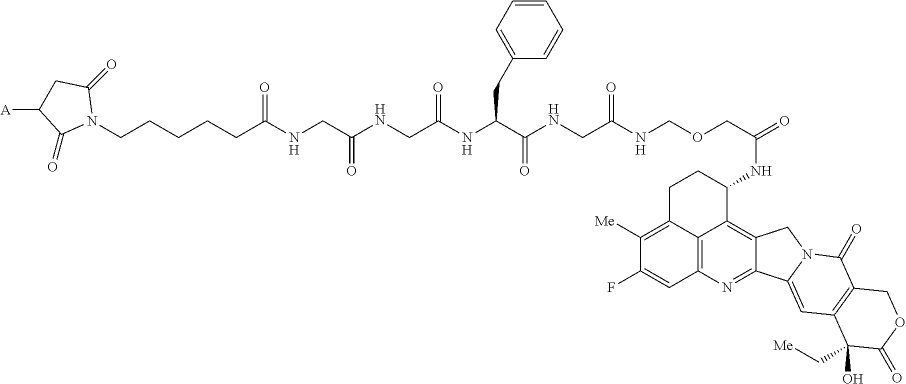

wherein A represents the connecting position to an antibody,
is conjugated to an antibody, preferably an anti-TROP2 or anti-HER2 antibody, via a thioether bond.

In the present disclosure, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present disclosure includes exatecan (IUPAC name: (1S,9S)-1-amino-9-ethyl-5-fluoro-1,2,3,9,12,15-hexahydro-9-hydroxy-4-methyl-10H,13H-benzo[de]pyrano[3′,4′:6,7] indolizino[1,2-b]quinolin-10,13-dione, (also expressed as chemical name: (1S,9S)-1-amino-9-ethyl-5-fluoro-2,3-dihydro-9-hydroxy-4-methyl-1H,12H-benzo[de]pyrano[3′,4′:6,7] indolizino[1,2-b]quinolin-10,13(9H,15H)-dione)), which is a topoisomerase I inhibitor, as a component. Exatecan is a camptothecin derivative having an antitumor effect, represented by the following formula:

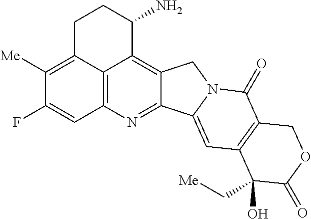 [SMILES: CC[C@@]1(O)C(=O)OCc2c1cc1n(c2=O)Cc2c-1nc1cc(F)c(C)c3c1c2[C@@H](N)CC3]

The antibody-drug conjugate used in the present disclosure can be also represented by the following formula:

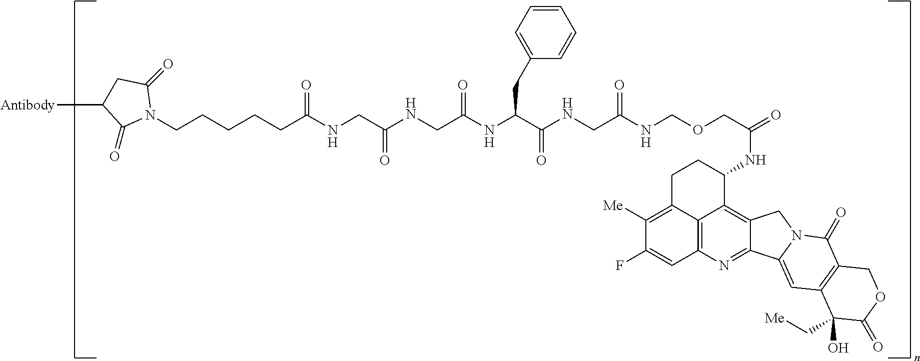

Here, the drug-linker is conjugated to an antibody (‘Antibody-’), preferably an anti-TROP2 or anti-HER2 antibody, via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the antibody-drug conjugate used in the present disclosure is cleaved at the linker portion to release a compound represented by the following formula:

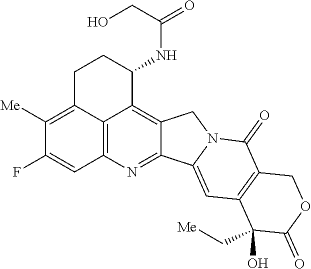 [SMILES: CC[C@@]1(O)C(=O)OCc2c1cc1n(c2=O)Cc2c-1nc1cc(F)c(C)c3c1c2[C@@H](NC(=O)CO)CC3]

### 2. Antibody in Antibody-Drug Conjugate

The antibody in the antibody-drug conjugate used in the present disclosure is preferably an anti-TROP2 or anti-HER2 antibody, and may be derived from any species, preferably from a human, a rat, a mouse, or a rabbit. In cases when the antibody is derived from species other than human species, it is preferably chimerized or humanized using a well-known technique. The antibody may be a polyclonal antibody or a monoclonal antibody and is preferably a monoclonal antibody.

The antibody in the antibody-drug conjugate used in the present disclosure is an antibody preferably having a characteristic of being capable of targeting cancer cells, and is preferably an antibody possessing, for example, a property of recognizing a cancer cell, a property of binding to a cancer cell, a property of internalizing in a cancer cell, and/or cytocidal activity against cancer cells.

The binding activity of the antibody against cancer cells can be confirmed using flow cytometry. The internalization of the antibody into cancer cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282, December 2004), or (3) a Mab-ZAP assay using an immunotoxin binding to the therapeutic antibody wherein the toxin is released upon incorporation into cells to inhibit cell growth (Bio Techniques 28: 162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining change in the cancer cell.

Since the compound conjugated in the antibody-drug conjugate exerts an antitumor effect, it is preferred but not essential that the antibody itself should have an antitumor effect. For the purpose of specifically and selectively exerting the cytotoxic activity of the antitumor compound against cancer cells, it is important and also preferred that the antibody should have the property of internalizing to migrate into cancer cells.

The antibody (preferably anti-TROP2 or anti-HER2 antibody) in the antibody-drug conjugate used in the present disclosure can be obtained by a procedure known in the art. For example, the antibody of the present disclosure can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen are fused with myeloma cells according to a method known in the art (e.g., Kohler and Milstein, Nature (1975) 256, p. 495-497; and Kennet, R. ed., Monoclonal Antibodies, p. 365-367, Plenum Press, N.Y. (1980)) to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The antibody (preferably anti-TROP2 or anti-HER2 antibody) in the antibody-drug conjugate used the present disclosure is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), and an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002)43 (7), p. 2301-2308; Carmen, S. et. al., Briefings in Functional Genomics and Proteomics (2002), 1(2), p. 189-203; Siriwardena, D. et. al., Ophthalmology (2002) 109(3), p. 427-431, etc.) can be exemplified.

In the antibody in the antibody-drug conjugate used in present invention, modified variants of the antibody are also included. The modified variant refers to a variant obtained by subjecting the antibody according to the present disclosure to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present disclosure, for example, an enzyme-labeled antibody, a fluorescence-labeled antibody, and an affinity-labeled antibody are also included in the meaning of the modified variant. Such a modified variant of the antibody according to the present disclosure is useful for improving the stability and blood retention of the antibody, reducing the antigenicity thereof, detecting or isolating an antibody or an antigen, and so on.

Further, by regulating the modification of a glycan which is linked to the antibody according to the present disclosure (glycosylation, defucosylation, etc.), it is possible to enhance antibody-dependent cellular cytotoxic activity. As the technique for regulating the modification of a glycan of antibodies, those disclosed in WO99/54342, WO00/61739, WO02/31140, WO2007/133855, WO2013/120066, etc. are known. However, the technique is not limited thereto. In the antibody (preferably anti-TROP2 or anti-HER2 antibody) according to the present disclosure, antibodies in which the modification of a glycan is regulated are also included.

It is known that a lysine residue at the carboxyl terminus of the heavy chain of an antibody produced in a cultured mammalian cell is deleted (Journal of Chromatography A, 705: 129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360: 75-83 (2007)). However, such deletion and modification of the heavy chain sequence do not affect the antigen-binding affinity and the effector function (the activation of complement, antibody-dependent cellular cytotoxicity, etc.) of the antibody. Therefore, in the antibody (preferably anti-TROP2 or anti-HER2 antibody) according to the present disclosure, antibodies subjected to such modification and functional fragments of the antibody are also included, and deletion variants in which one or two amino acids have been deleted at the carboxyl terminus of the heavy chain, variants obtained by amidation of deletion variants (for example, a heavy chain in which the carboxyl terminal proline residue has been amidated), and the like are also included. The type of deletion variant having a deletion at the carboxyl terminus of the heavy chain of the antibody according to the present disclosure is not limited to the above variants as long as the antigen-binding affinity and the effector function are conserved. The two heavy chains constituting the antibody according to the present disclosure may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the present disclosure and the culture conditions; however, an antibody in which one amino acid residue at the carboxyl terminus has been deleted in both of the two heavy chains in the antibody according to the present disclosure can be exemplified as preferred.

As isotypes of the antibody (preferably anti-TROP2 or anti-HER2 antibody) according to the present disclosure, for example, IgG (IgG1, IgG2, IgG3, IgG4) can be exemplified, and IgG1 or IgG2 can be exemplified as preferred.

Antibodies applicable to production of the antibody-drug conjugate according to the present disclosure are not particularly limited to any particular antigen. However, an anti-TROP2 antibody and an anti-HER2 antibody can be exemplified as preferred.

In the present disclosure, the term “anti-TROP2 antibody” refers to an antibody which binds specifically to TROP2 (TACSTD2: Tumor-associated calcium signal transducer 2; EGP-1), and preferably has an activity of internalization in TROP2-expressing cells by binding to TROP2.

Examples of the anti-TROP2 antibody include hTINA1-H1L1 (WO 2015/098099), and datopotamab can be exemplified as preferred.

In the present disclosure, the term “anti-HER2 antibody” refers to an antibody which specifically binds to HER2 (Human Epidermal Growth Factor Receptor Type 2; ErbB-2), and preferably has an activity of internalizing in HER2-expressing cells by binding to HER2.

Examples of the anti-HER2 antibody include trastuzumab (U.S. Pat. No. 5,821,337) and pertuzumab (WO01/00245), and trastuzumab can be exemplified as preferred.

### 3. Production of Antibody-Drug Conjugate

A drug-linker intermediate for use in production of the antibody-drug conjugate according to the present disclosure is represented by the following formula:

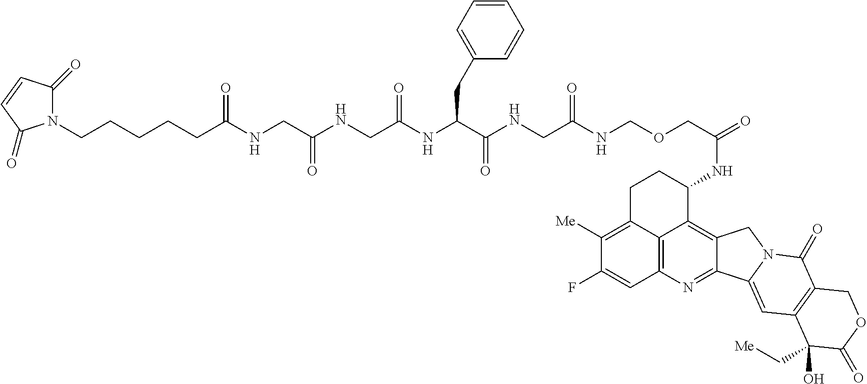 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl)hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7] indolizino[1,2-b]quinolin-1-yl]amino}-2-oxoethoxy)methyl]glycinamide, and can be produced with reference to descriptions in WO2014/057687, WO2015/098099, WO2015/115091, WO2015/155998, WO2019/044947 and so on.

The antibody-drug conjugate used in the present disclosure can be produced by reacting the above-described drug-linker intermediate and an antibody (preferably an anti-TROP2 or anti-HER2 antibody) having a thiol group (also referred to as a sulfhydryl group).

An antibody (preferably anti-TROP2 or anti-HER2 antibody) having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl)phosphine hydrochloride (TCEP) per interchain disulfide within the antibody and reacting with the antibody in a buffer solution containing a chelating agent such as ethylenediamine tetraacetic acid (EDTA), an antibody having a sulfhydryl group with partially or completely reduced interchain disulfides within the antibody can be obtained.

Further, by using 2 to 20 molar equivalents of the drug-linker intermediate per antibody (preferably anti-TROP2 or anti-HER2 antibody) having a sulfhydryl group, an antibody-drug conjugate in which 2 to 8 drug molecules are conjugated per antibody molecule can be produced.

The average number of conjugated drug molecules per antibody (preferably anti-TROP2 or anti-HER2 antibody) molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the antibody (preferably anti-TROP2 or anti-HER2 antibody) and the drug-linker intermediate and calculation of the average number of conjugated drug molecules per antibody molecule of the antibody-drug conjugate can be performed with reference to descriptions in WO2014/057687, WO2015/098099, WO2015/115091, WO2015/155998, WO2017/002776, WO2018/212136, and so on.

In the present invention, the term “anti-TROP2 antibody-drug conjugate” refers to an antibody-drug conjugate such that the antibody in the antibody-drug conjugate according to the invention is an anti-TROP2 antibody.

The anti-TROP2 antibody is preferably an antibody comprising a heavy chain comprising CDRH1 consisting of an amino acid sequence represented by SEQ ID NO: 3 [=an amino acid sequence consisting of amino acid residues 50 to 54 of SEQ ID NO: 1], CDRH2 consisting of an amino acid sequence represented by SEQ ID NO: 4 [=an amino acid sequence consisting of amino acid residues 69 to 85 of SEQ ID NO: 1], and CDRH3 consisting of an amino acid sequence represented by SEQ ID NO: 5 [=an amino acid sequence consisting of amino acid residues 118 to 129 of SEQ ID NO: 1], and a light chain comprising CDRL1 consisting of an amino acid sequence represented by SEQ ID NO: 6 [=an amino acid sequence consisting of amino acid residues 44 to 54 of SEQ ID NO: 2], CDRL2 consisting of an amino acid sequence represented by SEQ ID NO: 7 [=an amino acid sequence consisting of amino acid residues 70 to 76 of SEQ ID NO: 2], and CDRL3 consisting of an amino acid sequence represented by SEQ ID NO: 8 [=an amino acid sequence consisting of amino acid residues 109 to 117 of SEQ ID NO: 2],
more preferably an antibody comprising a heavy chain comprising a heavy chain variable region consisting of an amino acid sequence represented by SEQ ID NO: 9 [=an amino acid sequence consisting of amino acid residues 20 to 140 of SEQ ID NO: 1], and a light chain comprising a light chain variable region consisting of an amino acid sequence represented by SEQ ID NO: 10 [=an amino acid sequence consisting of amino acid residues 21 to 129 of SEQ ID NO: 2], and
even more preferably an antibody comprising a heavy chain consisting of an amino acid sequence represented by SEQ ID NO: 12 [=an amino acid sequence consisting of amino acid residues 20 to 470 of SEQ ID NO: 1] and a light chain consisting of an amino acid sequence represented by SEQ ID NO: 13 [=amino acid residues 21 to 234 of SEQ ID NO: 2], or an antibody comprising a heavy chain consisting of an amino acid sequence represented by SEQ ID NO: 11 [=an amino acid sequence consisting of amino acid residues 20 to 469 of SEQ ID NO: 1] and a light chain consisting of an amino acid sequence represented by SEQ ID NO: 13 [=amino acid residues 21 to 234 of SEQ ID NO: 2].

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in WO 2015/098099 and WO 2017/002776.

In preferred embodiments, the anti-TROP2 antibody-drug conjugate is datopotamab deruxtecan (DS-1062).

In the present disclosure, the term “anti-HER2 antibody-drug conjugate” refers to an antibody-drug conjugate such that the antibody in the antibody-drug conjugate according to the present disclosure is an anti-HER2 antibody.

The anti-HER2 antibody is preferably an antibody comprising a heavy chain comprising CDRH1 consisting of an amino acid sequence consisting of amino acid residues 26 to 33 of SEQ ID NO: 14, CDRH2 consisting of an amino acid sequence consisting of amino acid residues 51 to 58 of SEQ ID NO: 14 and CDRH3 consisting of an amino acid sequence consisting of amino acid residues 97 to 109 of SEQ ID NO: 14, and a light chain comprising CDRL1 consisting of an amino acid sequence consisting of amino acid residues 27 to 32 of SEQ ID NO: 15, CDRL2 consisting of an amino acid sequence consisting of amino acid residues 50 to 52 of SEQ ID NO: 15 and CDRL3 consisting of an amino acid sequence consisting of amino acid residues 89 to 97 of SEQ ID NO: 15, and more preferably an antibody comprising a heavy chain comprising a heavy chain variable region consisting of an amino acid sequence consisting of amino acid residues 1 to 120 of SEQ ID NO: 14 and a light chain comprising a light chain variable region consisting of an amino acid sequence consisting of amino acid residues 1 to 107 of SEQ ID NO: 15, and even more preferably an antibody comprising a heavy chain consisting of an amino acid sequence represented by SEQ ID NO: 14 and a light chain consisting of the amino acid sequence represented by SEQ ID NO: 15, or an antibody comprising a heavy chain consisting of amino acid residues 1 to 449 of SEQ ID NO: 14 and a light chain consisting of an amino acid sequence consisting of all amino acid residues 1 to 214 of SEQ ID NO: 15.

The average number of units of the drug-linker conjugated per antibody molecule in the anti-HER2 antibody-drug conjugate is preferably 2 to 8, more preferably 3 to 8, even more preferably 7 to 8, even more preferably 7.5 to 8, and even more preferably about 8.

The anti-HER2 antibody-drug conjugate used in the present disclosure can be produced with reference to descriptions in WO2015/115091 and so on.

In preferred embodiments, the anti-HER2 antibody-drug conjugate is trastuzumab deruxtecan (DS-8201).

### 4. Bispecific Checkpoint Inhibitor

In the present disclosure, the term “bispecific checkpoint inhibitor” refers to binding proteins that are bispecific. As used herein, bispecific binding proteins have binding specificities for at least two independent antigens (or targets) or different epitopes within the same antigen. Exemplary bispecific binding proteins may bind to two different epitopes of a target, or may bind two different targets. Other such binding proteins may combine a first target binding site with a second binding site for another target. In some aspects, the binding protein is a bispecific antibody.

In some aspects, bispecific antibodies provide additive and/or synergistic therapeutic effects derived from targeting two antigens simultaneously, with the administration of a single manufactured molecule.

In some aspects, the antibodies provided herein are monovalent bispecific antibodies (MBab). The monovalent bispecific antibody scaffolds described herein provide a superior platform for the generation of bispecific antibodies that fulfill all the benefits associated with bispecific antibodies while reducing the potential therapeutic risks mentioned above due to their monovalent nature. Furthermore, the MBabs provided herein are readily expressed, stable, and are likely to have low immunogenicity. As used herein, the term “monovalent bispecific”, which may be abbreviated “MBab”, refers to bispecific antibodies, where each arm can specifically bind to a different target antigen, and for a given pair of different target antigens (A and B), the MBab can bind to one of each. In certain aspects, monovalent bispecific antibodies can specifically bind to two independent antigens (or targets) or two independent epitopes on the same antigen. Typically, monovalent bispecific antibodies comprise two different variable regions. In some aspects, the binding affinity for the two independent antigens is about the same. In some aspects, the binding affinities for the two independent antigens are different.

The bispecific checkpoint inhibitor is preferably a bispecific binding protein that comprises a first binding domain that specifically binds to PD-1, and a second binding domain that specifically binds to CTLA-4 or TIGIT.

In a preferred embodiment, the bispecific binding protein comprises:
a) a first binding domain that specifically binds to PD-1, wherein the first binding domain comprises a heavy chain variable domain comprising a CDRH1 having the amino acid sequence of SEQ ID NO: 25, a CDRH2 having the amino acid sequence of SEQ ID NO: 26, and a CDRH3 having the amino acid sequence of SEQ ID NO: 27, and a light chain variable domain comprising a CDRL1 having the amino acid sequence of SEQ ID NO: 28, a CDRL2 having the amino acid sequence of SEQ ID NO: 29 and a CDRL3 having the amino acid sequence of SEQ ID NO: 30; and
b) a second binding domain that specifically binds to TIGIT, wherein the second binding domain comprises a heavy chain variable domain comprising a CDRH1 having the amino acid sequence of SEQ ID NO: 35, a CDRH2 having the amino acid sequence of SEQ ID NO: 36, and a CDRH3 having the amino acid sequence of SEQ ID NO: 37, and a light chain variable domain comprising a CDRL1 having the amino acid sequence of SEQ ID NO: 38, a CDRL2 having the amino acid sequence of SEQ ID NO: 39, and a CDRL3 having the amino acid sequence of SEQ ID NO: 40.

More preferably, the first binding domain that specifically binds to PD-1 comprises a heavy chain variable domain having the amino acid sequence of SEQ ID NO: 31 and a light chain variable domain having the amino acid sequence of SEQ ID NO: 33. In some aspects, the first binding domain that specifically binds to PD-1 comprises a heavy chain variable domain having an amino acid sequence that is at least 90, 91, 92, 93, 94, 95, 96, 97, 98, or 99% identical to the amino acid sequence of SEQ ID NO: 31. In some aspects, the first binding domain that specifically binds to PD-1 comprises a light chain variable domain having an amino acid sequence that is at least 90, 91, 92, 93, 94, 95, 96, 97, 98, or 99% identical to the amino acid sequence of SEQ ID NO: 33.

More preferably, the first binding domain that specifically binds to PD-1 comprises a heavy chain having the amino acid sequence of SEQ ID NO: 32 and a light chain having the amino acid sequence of SEQ ID NO: 34. In some aspects, the first binding domain that specifically binds to PD-1 comprises a heavy chain having an amino acid sequence that is at least 90, 91, 92, 93, 94, 95, 96, 97, 98, or 99% identical to the amino acid sequence of SEQ ID NO: 32. In some aspects, the first binding domain that specifically binds to PD-1 comprises a light chain having an amino acid sequence that is at least 90, 91, 92, 93, 94, 95, 96, 97, 98, or 99% identical to the amino acid sequence of SEQ ID NO: 34.

More preferably, the second binding domain that specifically binds TIGIT comprises a heavy chain variable domain having the amino acid sequence of SEQ ID NO: 41 and a light chain variable domain having the amino acid sequence of SEQ ID NO: 43. In some aspects, the second binding domain that specifically binds to TIGIT comprises a heavy chain variable domain having an amino acid sequence that is at least 90, 91, 92, 93, 94, 95, 96, 97, 98, or 99% identical to the amino acid sequence of SEQ ID NO: 41. In some aspects, the second binding domain that specifically binds to TIGIT comprises a light chain variable domain having an amino acid sequence that is at least 90, 91, 92, 93, 94, 95, 96, 97, 98, or 99% identical to the amino acid sequence of SEQ ID NO: 43.

More preferably, the second binding domain that specifically binds to TIGIT comprises a heavy chain having the amino sequence of SEQ ID NO: 42 and a light chain having the amino acid sequence of SEQ ID NO: 44. In some aspects, the second binding domain that specifically binds to TIGIT comprises a heavy chain having an amino acid sequence that is at least 90, 91, 92, 93, 94, 95, 96, 97, 98, or 99% identical to the amino acid sequence of SEQ ID NO: 42. In some aspects, the second binding domain that specifically binds to TIGIT comprises a light chain having an amino acid sequence that is at least 90, 91, 92, 93, 94, 95, 96, 97, 98, or 99% identical to the amino acid sequence of SEQ ID NO: 44.

In a particularly preferred embodiment, the bispecific binding protein is an antibody that comprises:
(a) a first binding domain that specifically binds to PD-1, comprising a heavy chain having the amino acid sequence of SEQ ID NO: 32 and a light chain having the amino acid sequence of SEQ ID NO: 34; and
(b) a second binding domain that specifically binds to TIGIT, comprising a heavy chain having the amino sequence of SEQ ID NO: 42 and a light chain having the amino acid sequence of SEQ ID NO: 44.

In another preferred embodiment, the bispecific binding protein comprises:
a) a first binding domain that specifically binds to PD-1, wherein the first binding domain comprises a heavy chain variable domain comprising a CDRH1 having the amino acid sequence of SEQ ID NO: 52, a CDRH2 having the amino acid sequence of SEQ ID NO: 53, and a CDRH3 having the amino acid sequence of SEQ ID NO: 54, and a light chain variable domain comprising a CDRL1 having the amino acid sequence of SEQ ID NO: 49, a CDRL2 having the amino acid sequence of SEQ ID NO: 50 and a CDRL3 having the amino acid sequence of SEQ ID NO: 51; and
b) a second binding domain that specifically binds to CTLA-4, wherein the second binding domain comprises a heavy chain variable domain comprising a CDRH1 having the amino acid sequence of SEQ ID NO: 58, a CDRH2 having the amino acid sequence of SEQ ID NO: 59, and a CDRH3 having the amino acid sequence of SEQ ID NO: 60, and a light chain variable domain comprising a CDRL1 having the amino acid sequence of SEQ ID NO: 55, a CDRL2 having the amino acid sequence of SEQ ID NO: 56, and a CDRL3 having the amino acid sequence of SEQ ID NO: 57.

More preferably, the first binding domain that specifically binds to PD-1 comprises a heavy chain having the amino acid sequence of SEQ ID NO: 46 and a light chain having the amino acid sequence of SEQ ID NO: 45. In some aspects, the first binding domain that specifically binds to PD-1 comprises a heavy chain having an amino acid sequence that is at least 90, 91, 92, 93, 94, 95, 96, 97, 98, or 99% identical to the amino acid sequence of SEQ ID NO: 46. In some aspects, the first binding domain that specifically binds to PD-1 comprises a light chain having an amino acid sequence that is at least 90, 91, 92, 93, 94, 95, 96, 97, 98, or 99% identical to the amino acid sequence of SEQ ID NO: 45.

More preferably, the second binding domain that specifically binds to CTLA-4 comprises a heavy chain having the amino sequence of SEQ ID NO: 48 and a light chain having the amino acid sequence of SEQ ID NO: 47. In some aspects, the second binding domain that specifically binds to CTLA-4 comprises a heavy chain having an amino acid sequence that is at least 90, 91, 92, 93, 94, 95, 96, 97, 98, or 99% identical to the amino acid sequence of SEQ ID NO: 48. In some aspects, the second binding domain that specifically binds to CTLA-4 comprises a light chain having an amino acid sequence that is at least 90, 91, 92, 93, 94, 95, 96, 97, 98, or 99% identical to the amino acid sequence of SEQ ID NO: 47.

In a particularly preferred embodiment, the bispecific binding protein is an antibody that comprises:
(a) a first binding domain that specifically binds to PD-1, comprising a heavy chain having the amino acid sequence of SEQ ID NO: 46 and a light chain having the amino acid sequence of SEQ ID NO: 45; and
(b) a second binding domain that specifically binds to CTLA-4, comprising a heavy chain having the amino sequence of SEQ ID NO: 48 and a light chain having the amino acid sequence of SEQ ID NO: 47.

In a further particularly preferred embodiment, the bispecific binding protein is MEDI5752. As used herein, the term “MEDI5752” or “volrustomig” refers to an anti-PD-1/CTLA-4 bispecific antibody that comprises the light chain of SEQ ID NO: 45 and the heavy chain of SEQ ID NO: 46 (PD-1) and the light chain of SEQ ID NO: 47 and the heavy chain of SEQ ID NO: 48 (CTLA-4). MEDI5752 is disclosed in U.S. Pat. No. 10,457,732, which is incorporated by reference herein in its entirety.

In some aspects, the light chain constant region of the bispecific binding protein is a kappa chain. In some aspects, the light chain constant region is a lambda chain.

In some aspects, the bispecific binding protein comprises an variant Fc region comprising at least one substitution selected from 221K, 221Y, 225E, 225K, 225W, 228P, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 2621, 262A, 262T, 262E, 2631, 263A, 263T, 263M, 264L, 2641, 264W, 264T, 264R, 264F, 264M, 264Y, 264E, 265A, 265G, 265N, 265Q, 265Y, 265F, 265V, 2651, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 296G, 297S, 297D, 297E, 298A, 298H, 298I, 298T, 298F, 299I, 299L, 299A, 299S, 299V, 299H, 299F, 299E, 305I, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat.

In some aspects, the variant Fc region comprises one or more modifications at positions selected from 428 and 434 as numbered by the EU index as set forth in Kabat. In some aspects, the variant Fc region comprises one or more amino acid substitutions at positions selected from 428 and 434 as numbered by the EU index as set forth in Kabat. In some aspects, the variant Fc region comprises one or more amino acid substitutions selected from 428L, 428F, 434A, 434W, and 434Y.

Fc region engineering is widely used in the art to extend the half-life of therapeutic antibodies and protect from degradation in vivo. In some aspects, the Fc region of an IgG antibody or antigen-binding fragment can be modified in order to increase the affinity of the IgG molecule for the Fc Receptor-neonate (FcRn), which mediates IgG catabolism and protects IgG molecules from degradation.

In some aspects, the antibody or antigen-binding fragment thereof comprises an Fc region that has been engineered to improve half-life.

In some aspects, the Fc region is aglycosylated. In some aspects, the Fc region is deglycosylated. In some aspects, the Fc region has reduced fucosylation or is afucosylated.

In some aspects, the Fc variant antibody or binding fragment thereof has an increased binding affinity for FcRn.

The triple mutation (TM) L234F/L235E/P331S (according to European Union numbering convention; Sazinsky et al. Proc Natl Acad Sci USA, 105:20167-20172 (2008)) in the heavy chain constant region can significantly reduce IgG effector function. In some aspects, the antibody or antigen-binding fragment thereof comprises an Fc region with a L234F/L235E/P331S triple mutation (TM).

In some aspects, the Fc variant antibody or binding fragment thereof has reduced complement dependent cytotoxicity (CDC) when administered in vivo. In some aspects, the Fc variant antibody or binding fragment thereof has reduced CDC compared to an antibody or binding variant thereof that contains a wild-type Fc region. In some aspects, the Fc variant antibody or binding fragment thereof does not trigger CDC when administered in vivo. In some aspects, the Fc variant antibody or binding fragment thereof causes reduced CDC when administered in vivo. In some aspects, the Fc variant antibody or binding fragment thereof having reduced CDC activity or no CDC activity comprises the triple mutation (L234F/L235E/P331S) in the variant Fc region.

In some aspects, the Fc variant antibody or binding fragment thereof has reduced antibody dependent cellular cytotoxicity (ADCC) when administered in vivo. In some aspects, the Fc variant antibody or binding fragment thereof has reduced ADCC compared to an antibody or binding variant thereof that contains a wild-type Fc region. In some aspects, the Fc variant antibody or binding fragment thereof does not trigger ADCC when administered in vivo. In some aspects, the Fc variant antibody or binding fragment thereof causes reduced ADCC when administered in vivo. In some aspects, the Fc variant antibody or binding fragment thereof having reduced ADCC activity or no ADCC activity comprises the triple mutation (L234F/L235E/P331S) in the variant Fc region.

In some aspects, the antibody or binding fragment thereof having reduced CDC activity has reduced toxicity when administered to a subject. In some aspects, the antibody or binding fragment thereof having reduced ADCC activity has reduced toxicity when administered to a subject.

The term “Fc domain” as used herein encompasses native Fc and Fc variants and sequences as defined above. As with Fc variants and native Fc molecules, the term “Fc domain” includes molecules in monomeric or multimeric form, whether digested from whole antibody or produced by other means.

The term “native Fc” as used herein refers to a molecule comprising the sequence of a non-antigen binding fragment resulting from digestion of an antibody or produced by other means, whether in monomeric or multimeric form, and can contain the hinge region. The original immunoglobulin source of the native Fc is preferably of human origin and can be any of the immunoglobulins. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc” as used herein is generic to the monomeric, dimeric, and multimeric forms.

In some aspects, the Fc region is or includes a domain that is one or more of an Fc region from an IgG1, IgG2, IgG3, IgG4, IgA, IgM, IgE, or IgD. In some aspects, the antibody is an IgG1 antibody.

The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed or mutated to produce an Fc variant to alter certain residues that provide structural features or biological activity that are not required for the binding proteins of the disclosure. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3)N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

To improve the yields of the binding proteins, the CH3 domains can be altered by the “knob-into-holes” technology which is described in detail with several exemplary molecules in, for example, WO 96/027011, Ridgway et al., 1996, Protein Eng. 9: 617-21; and Merchant et al., 1998, Nat. Biotechnol. 16: 677-81. Specifically, the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge further stabilizes the heterodimers (Merchant et al., 1998; Atwell et al., 1997, J. Mol. Biol. 270: 26-35) and increases the yield.

In some aspects, the binding proteins have a “DuetMab” format. DuetMab has the following basic structure: an Fc region having a modified heavy chain, wherein the CH1 region of the modified heavy chain has a substitution of a native cysteine to a non-cysteine amino acid, and a substitution of a native non-cysteine amino acid to a cysteine amino acid; a modified corresponding light chain, where the CL region of the modified light chain also has a substitution of a native cysteine to a non-cysteine amino acid, and a substitution of a native non-cysteine amino acid to a cysteine amino acid; a second Fc region having a second heavy chain; and second corresponding modified light chain, where the modified heavy chain is directly linked to the corresponding modified light chain, and on a separate target binding arm, the second heavy chain is directly linked to the second corresponding light chain, and where the substituted cysteine of the modified heavy chain, resulting from the substitution of the native non-cysteine amino acid to the cysteine amino acid, and the substituted cysteine of the modified corresponding light chain, resulting from the substitution of the native non-cysteine amino acid to the cysteine amino acid, can form a disulphide bond. Disclosure related to DuetMab can found, for example, in U.S. Pat. No. 9,527,927, incorporated herein by reference in its entirety.

The term “KD” as used herein, refers to the dissociation constant (KD=[A]×[B]/[AB]) of the interaction between a binding protein the disclosure and an antigen target and has the units of moles/liter. A binding protein of the disclosure typically has a dissociation constant (KD) of 10-5 to 10-12 moles/liter or less, or 10-7 to 10-12 moles/liter or less, or 10-3 to 10-12 moles/liter, and/or with a binding affinity of at least 107 M-1, or at least 108 M-1, or at least 109 M-1, or at least 1012 M-1. Any KD value greater than 10-4 moles/liter is generally considered to indicate non-specific binding. Therefore, the lower the KD value, the greater the affinity. In some aspects, a binding protein of the disclosure will bind to a desired antigen with an affinity less than 500 nM, or less than 200 nM, or less than 10 nM, or less than 500 pM. High affinity or very strong binding is often associated with greater efficacy, but it is not always the case that the greater the affinity the greater the efficacy.

The dissociation constant (KD) can be determined, for example, by surface plasmon resonance (SPR). Generally, surface plasmon resonance analysis measures real-time binding interactions (both on rate and off rate) between a ligand (a target antigen on a biosensor matrix) and an analyte by surface plasmon resonance using, for example, the BIAcore® system (Pharmacia Biosensor; Piscataway, NJ). Surface plasmon analysis can also be performed by immobilizing the analyte and presenting the ligand. Specific binding of binding protein of the disclosure to an antigen or antigenic determinant can also be determined in any suitable manner known in the art, including, for example, Scatchard analysis and/or competitive binding assays, such as radioimmunoassays (RIA), enzyme linked immunosorbent assays (ELISA), enzyme immunoassays (EIA), and sandwich competition assays.

In one aspect, the equilibrium dissociation constant (KD) of an interaction of a bispecific binding protein as described herein with human TIGIT is less than or equal to about 15 pM. In one aspect, the KD of an interaction of a bispecific binding protein as described herein with human TIGIT is less than or equal to about 9 pM. In one aspect, the KD of an interaction of a bispecific binding protein as described herein with human TIGIT is less than or equal to about 15, 14, 13, 12, 11, 10, 9, or 8 pM.

In one aspect, the KD of an interaction of a bispecific binding protein as described herein with human TIGIT is from about 9 pM to about 15 pM. In one aspect, the KD of an interaction of a bispecific binding protein as described herein with human TIGIT is from about 10 pM to about 15 pM. In one aspect, the KD of an interaction of a bispecific binding protein as described herein with human TIGIT is from about 11 pM to about 15 pM. In one aspect, the KD of an interaction of a bispecific binding protein as described herein with human TIGIT is from about 12 pM to about 15 pM. In one aspect, the KD of an interaction of a bispecific binding protein as described herein with human TIGIT is from about 13 pM to about 15 pM. In one aspect, the KD of an interaction of a bispecific binding protein as described herein with human TIGIT is from about 14 pM to about 15 pM.

In one aspect, the KD of an interaction of a bispecific binding protein as described herein with human PD-L1 is less than or equal to about 0.4 nM. In one aspect, the KD of an interaction of a bispecific binding protein as described herein with human PD-L1 is from about 0.2 nM to about 0.5 nM. In one aspect, the KD of an interaction of a bispecific binding protein as described herein with human PD-L1 is from about 0.3 nM to about 0.5 nM. In one aspect, the KD of an interaction of a bispecific binding protein as described herein with human PD-L1 is from about 0.4 nM to about 0.5 nM.

### 5. Combination of Antibody-Drug Conjugate and Bispecific Checkpoint Inhibitor

In a first combination embodiment of the disclosure, the antibody-drug conjugate which is combined with the bispecific checkpoint inhibitor is an antibody-drug conjugate in which the antibody is an anti-TROP2 antibody.

In an embodiment of the first combination embodiment described above, the anti-TROP2 antibody comprises a heavy chain comprising CDRH1 consisting of an amino acid sequence represented by SEQ ID NO: 3 [=amino acid residues 50 to 54 of SEQ ID NO: 1], CDRH2 consisting of an amino acid sequence represented by SEQ ID NO: 4 [=amino acid residues 69 to 85 of SEQ ID NO: 1] and CDRH3 consisting of an amino acid sequence represented by SEQ ID NO: 5 [=amino acid residues 118 to 129 of SEQ ID NO: 1], and a light chain comprising CDRL1 consisting of an amino acid sequence represented by SEQ ID NO: 6 [=amino acid residues 44 to 54 of SEQ ID NO: 2], CDRL2 consisting of an amino acid sequence represented by SEQ ID NO: 7 [=amino acid residues 70 to 76 of SEQ ID NO: 2] and CDRL3 consisting of an amino acid sequence represented by SEQ ID NO: 8 [=amino acid residues 109 to 117 of SEQ ID NO: 2]. In another embodiment of the first combination embodiment described above, the anti-TROP2 antibody comprises a heavy chain comprising a heavy chain variable region consisting of an amino acid sequence represented by SEQ ID NO: 9 [=amino acid residues 20 to 140 of SEQ ID NO: 1] and a light chain comprising a light chain variable region consisting of an amino acid sequence represented by SEQ ID NO: 10 [=amino acid residues 21 to 129 of SEQ ID NO: 2]. In another embodiment of the first combination embodiment described above, the anti-TROP2 antibody comprises a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]. In another embodiment of the first combination embodiment described above, the anti-TROP2 antibody comprises a heavy chain consisting of an amino acid sequence represented by SEQ ID NO: 11 [=amino acid residues 20 to 469 of SEQ ID NO: 1] and a light chain consisting of an amino acid sequence represented by SEQ ID NO: 13 [=amino acid residues 21 to 234 of SEQ ID NO: 2]. In another embodiment of the first combination embodiment described above, the anti-TROP2 antibody is datopotamab deruxtecan (DS-1062).

In a second combination embodiment of the disclosure, the antibody-drug conjugate which is combined with the bispecific checkpoint inhibitor is an antibody-drug conjugate in which the antibody is an anti-HER2 antibody.

In an embodiment of the second combination embodiment described above, the anti-HER2 antibody comprises a heavy chain comprising CDRH1 consisting of an amino acid sequence represented by SEQ ID NO: 16, CDRH2 consisting of an amino acid sequence represented by SEQ ID NO: 17 and CDRH3 consisting of an amino acid sequence represented by SEQ ID NO: 18, and a light chain comprising CDRL1 consisting of an amino acid sequence represented by SEQ ID NO: 19, CDRL2 consisting of an amino acid sequence consisting of amino acid residues 1 to 3 of SEQ ID NO: 20 and CDRL3 consisting of an amino acid sequence represented by SEQ ID NO: 21. In another embodiment of the second combination embodiment described above, the anti-HER2 antibody comprises a heavy chain comprising a heavy chain variable region consisting of an amino acid sequence represented by SEQ ID NO: 22 and a light chain comprising a light chain variable region consisting of an amino acid sequence represented by SEQ ID NO: 23. In another embodiment of the second combination embodiment described above, the anti-HER2 antibody comprises a heavy chain consisting of an amino acid sequence represented by SEQ ID NO: 14 and a light chain consisting of an amino acid sequence represented by SEQ ID NO: 15. In another embodiment of the second combination embodiment described above, the anti-HER2 antibody comprises a heavy chain consisting of an amino acid sequence represented by SEQ ID NO: 24 and a light chain consisting of an amino acid sequence represented by SEQ ID NO: 15. In another embodiment of the second combination embodiment described above, the anti-HER2 antibody is trastuzumab deruxtecan (DS-8201).

In a particularly preferred embodiment of the first combination embodiment described above, the anti-TROP2 antibody-drug conjugate is datopotamab deruxtecan (DS-1062) and the bispecific checkpoint inhibitor is an anti-PD-1/TIGIT bispecific antibody in which a first binding domain that specifically binds to PD-1 comprises a heavy chain having the amino acid sequence of SEQ ID NO: 32 and a light chain having the amino acid sequence of SEQ ID NO: 34, and in which a second binding domain that specifically binds TIGIT comprises a heavy chain variable domain having the amino acid sequence of SEQ ID NO: 41 and a light chain variable domain having the amino acid sequence of SEQ ID NO: 43.

In another particularly preferred embodiment of the first combination embodiment described above, the anti-TROP2 antibody-drug conjugate is datopotamab deruxtecan (DS-1062) and the bispecific checkpoint inhibitor is an anti-PD-1/CTLA-4 bispecific antibody in which a first binding domain that specifically binds to PD-1 comprises a heavy chain having the amino acid sequence of SEQ ID NO: 46 and a light chain having the amino acid sequence of SEQ ID NO: 45, and in which a second binding domain that specifically binds CTLA-4 comprises a heavy chain variable domain having the amino acid sequence of SEQ ID NO: 48 and a light chain variable domain having the amino acid sequence of SEQ ID NO: 47.

In a particularly preferred embodiment of the second combination embodiment described above, the anti-HER2 antibody-drug conjugate is trastuzumab deruxtecan (DS-8201) and the bispecific checkpoint inhibitor is an anti-PD-1/TIGIT bispecific antibody in which a first binding domain that specifically binds to PD-1 comprises a heavy chain having the amino acid sequence of SEQ ID NO: 32 and a light chain having the amino acid sequence of SEQ ID NO: 34, and in which a second binding domain that specifically binds TIGIT comprises a heavy chain variable domain having the amino acid sequence of SEQ ID NO: 41 and a light chain variable domain having the amino acid sequence of SEQ ID NO: 43.

In another particularly preferred embodiment of the second combination embodiment described above, the anti-HER2 antibody-drug conjugate is trastuzumab deruxtecan (DS-8201) and the bispecific checkpoint inhibitor is an anti-PD-1/CTLA-4 bispecific antibody in which a first binding domain that specifically binds to PD-1 comprises a heavy chain having the amino acid sequence of SEQ ID NO: 46 and a light chain having the amino acid sequence of SEQ ID NO: 45, and in which a second binding domain that specifically binds CTLA-4 comprises a heavy chain variable domain having the amino acid sequence of SEQ ID NO: 48 and a light chain variable domain having the amino acid sequence of SEQ ID NO: 47.

In some aspects of the above combination embodiments, the antibody-drug conjugate and bispecific checkpoint inhibitor are administered in further combination with one or more chemotherapeutic agents. In some aspects, the chemotherapeutic agent is carboplatin. In some aspects, the chemotherapeutic agent is a fluoropyrimidine (e.g., fluorouracil, 5-FU).

### 6. Therapeutic Combined Use and Method

Described in the following are a pharmaceutical product and a therapeutic use and method wherein the antibody-drug conjugate (preferably anti-TROP2 or anti-HER2 antibody-drug conjugate) according to the present disclosure and a bispecific checkpoint inhibitor are administered in combination.

The pharmaceutical product and therapeutic use and method of the present disclosure may be characterized in that the antibody-drug conjugate and the bispecific checkpoint inhibitor are separately contained as active components in different formulations, and are administered simultaneously or at different times, or characterized in that the antibody-drug conjugate and the bispecific checkpoint inhibitor are contained as active components in a single formulation and administered.

In the pharmaceutical product and therapeutic method of the present disclosure, a single bispecific checkpoint inhibitor used in the present disclosure can be administered in combination with the antibody-drug conjugate, or two or more different bispecific checkpoint inhibitors can be administered in combination with the antibody-drug conjugate.

The pharmaceutical product and therapeutic method of the present disclosure can be used for treating cancer, and can be preferably used for treating at least one cancer selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, melanoma, cervical cancer, uterine cancer, testicular cancer, and renal cell carcinoma.

The presence or absence of tumor markers such as HER2 or TROP2 tumor markers can be determined, for example, by collecting tumor tissue from a cancer patient to prepare a formalin-fixed, paraffin-embedded (FFPE) specimen and subjecting the specimen to a test for gene products (proteins), for example, with an immunohistochemical (IHC) method, a flow cytometer, or Western blotting, or to a test for gene transcription, for example, with an in situ hybridization (ISH) method, a quantitative PCR method (q-PCR), or microarray analysis, or by collecting cell-free circulating tumor DNA (ctDNA) from a cancer patient and subjecting the ctDNA to a test with a method such as next-generation sequencing (NGS).

The pharmaceutical product and therapeutic method of the present disclosure, when comprising anti-HER2 antibody-drug conjugate, can be used for HER2-expressing cancer, which may be HER2-overexpressing cancer (high or moderate) or may be HER2 low-expressing cancer.

In the present disclosure, the term “HER2-overexpressing cancer” is not particularly limited as long as it is recognized as HER2-overexpressing cancer by those skilled in the art. Preferred examples of the HER2-overexpressing cancer can include cancer given a score of 3+ for the expression of HER2 in an IHC method, and cancer given a score of 2+ for the expression of HER2 in an IHC method and determined as positive for the expression of HER2 in an in situ hybridization method (ISH). The in situ hybridization method of the present disclosure includes a fluorescence in situ hybridization method (FISH) and a dual color in situ hybridization method (DISH).

In the present disclosure, the term “HER2 low-expressing cancer” is not particularly limited as long as it is recognized as HER2 low-expressing cancer by those skilled in the art. Preferred examples of the HER2 low-expressing cancer can include cancer given a score of 2+ for the expression of HER2 in an IHC method and determined as negative for the expression of HER2 in an in situ hybridization method, and cancer given a score of 1+ for the expression of HER2 in an IHC method.

The method for scoring the degree of HER2 expression by the IHC method, or the method for determining positivity or negativity to HER2 expression by the in situ hybridization method is not particularly limited as long as it is recognized by those skilled in the art. Examples of the method can include a method described in the 4th edition of the guidelines for HER2 testing, breast cancer (developed by the Japanese Pathology Board for Optimal Use of HER2 for Breast Cancer).

The cancer, particularly in regard to the treatment of breast cancer, may be HER2-overexpressing (high or moderate) or low-expressing breast cancer, or triple-negative breast cancer, and/or may have a HER2 status score of IHC 3+, IHC 2+, IHC 1+ or IHC>0 and <1+.

In some aspects, the methods of the present disclosure comprise identifying a patient as having a PD-L1 positive tumor prior to the administration of the bispecific checkpoint inhibitor.

In some aspects, the PD-L1 positive tumor comprises at least about 1%, at least about 2%, at least about 3%, at least about 4%, at least about 5%, at least about 7%, at least about 10%, at least about 15%, at least about 20%, at least about 25%, at least about 30%, at least about 40%, at least about 50%, at least about 60%, at least about 70%, at least about 80%, at least about 90%, or 100% cells expressing PD-L1.

In some aspects, PD-L1 expression is determined by receiving the results of an assay capable of determining PD-L1 expression. In some aspects, the assay capable of determining PD-L1 expression is a Ventana PD-L1 (SP263) IHC assay, a 22C3 PharmDx assay, or a 28-8 PharmDx assay.

In some aspects, the methods of the present disclosure comprise administering an antibody-drug conjugate and an anti-PD-1/CTLA-4 bispecific antibody to a patient whose tumor comprises less than 50% PD-L1 expressing cells. In some aspects, the methods of the present disclosure comprise administering an antibody-drug conjugate, an anti-PD-1/CTLA-4 bispecific antibody, and carboplatin to a patient whose tumor comprises less than 50% PD-L1 expressing cells.

In some aspects, the methods of the present disclosure comprise administering an antibody-drug conjugate and an anti-PD-1/TIGIT bispecific antibody to a patient whose tumor comprises at least 50% PD-L1 expressing cells. In some aspects, the methods of the present disclosure comprise administering an antibody-drug conjugate, an anti-PD-1/TIGIT bispecific antibody, and carboplatin to a patient whose tumor comprises less than 50% PD-L1 expressing cells.

The pharmaceutical product and therapeutic method of the present disclosure can be preferably used for a mammal, but are more preferably used for a human.

The antitumor effect of the pharmaceutical product and therapeutic method of the present disclosure can be confirmed by transplanting cancer cells to a test subject animal to prepare a model and measuring reduction in tumor volume or life-prolonging effect by application of the pharmaceutical product and therapeutic method of the present disclosure. And then, the effect of combined use of the antibody-drug conjugate used in the present disclosure and a bispecific checkpoint inhibitor can be confirmed by comparing antitumor effect with single administration of the antibody-drug conjugate used in the present disclosure and that of the bispecific checkpoint inhibitor.

The antitumor effect of the pharmaceutical product and therapeutic method of the present disclosure can be confirmed in a clinical trial using any of an evaluation method with Response Evaluation Criteria in Solid Tumors (RECIST), a WHO evaluation method, a Macdonald evaluation method, body weight measurement, and other approaches, and can be determined on the basis of indexes of complete response (CR), partial response (PR); progressive disease (PD), objective response rate (ORR), duration of response (DoR), progression-free survival (PFS), overall survival (OS), and so on.

By using the above methods, the superiority in antitumor effect of the pharmaceutical product and therapeutic method of the present disclosure to existing pharmaceutical products and therapeutic methods for cancer treatment can be confirmed.

The pharmaceutical product and therapeutic method of the present disclosure can delay development of cancer cells, inhibit growth thereof, and further kill cancer cells. These effects can allow cancer patients to be free from symptoms caused by cancer or achieve improvement in quality of life (QOL) of cancer patients and attain a therapeutic effect by sustaining the lives of the cancer patients. Even if the pharmaceutical product and therapeutic method of the present disclosure do not accomplish killing cancer cells, they can achieve higher QOL of cancer patients while achieving longer-term survival, by inhibiting or controlling the growth of cancer cells.

The pharmaceutical product of the present disclosure can be expected to exert a therapeutic effect by application as systemic therapy to patients, and additionally, by local application to cancer tissues.

The pharmaceutical product and therapeutic method of the present disclosure, in another aspect, provides for use as an adjuct in cancer therapy with ionizing radiation or other chemotherapeutic agents. For example, in the treatment of cancer, the treatment may comprise administering to a subject in need of treatment a therapeutically-effective amount of the pharmaceutical product, simultaneously or sequentially with ionizing radiation or other chemotherapeutic agents.

The pharmaceutical product and therapeutic method of the present disclosure can be used as adjuvant chemotherapy combined with surgery operation. The pharmaceutical product of the present disclosure may be administered for the purpose of reducing tumor size before surgical operation (referred to as preoperative adjuvant chemotherapy or neoadjuvant therapy), or may be administered for the purpose of preventing recurrence of tumor after surgical operation (referred to as postoperative adjuvant chemotherapy or adjuvant therapy).

In some embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535). BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol Med., 8 (12), 571-6, (2002)). The association of BRCA1 and/or BRCA2 mutations with breast cancer is well-characterised in the art (Radice, P. J., Exp Clin Cancer Res., 21(3 Suppl), 9-12 (2002)). Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast, ovary, pancreas, prostate, hematological, gastrointestinal and lung cancer. In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Chappnis, P. O. and Foulkes, W. O., Cancer Treat Res, 107, 29-59 (2002); Janatova M., et al., Neoplasma, 50(4), 246-505 (2003); Jancarkova, N., Ceska Gynekol., 68{1), 11-6 (2003)). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The pharmaceutical product of the present disclosure can be administered containing at least one pharmaceutically suitable ingredient. Pharmaceutically suitable ingredients can be suitably selected and applied from formulation additives or the like that are generally used in the art, in accordance with the dosage, administration concentration, or the like of the antibody-drug conjugate used in the present disclosure and a bispecific checkpoint inhibitor. The antibody-drug conjugate used in the present disclosure can be administered, for example, as a pharmaceutical product containing a buffer such as histidine buffer, a vehicle such as sucrose and trehalose, and a surfactant such as Polysorbates 80 and 20. The antibody-drug conjugate used in the pharmaceutical product of the present disclosure can be preferably used as an injection, can be more preferably used as an aqueous injection or a lyophilized injection, and can be even more preferably used as a lyophilized injection. In the case that the pharmaceutical product containing the antibody-drug conjugate used in the present disclosure is an aqueous injection, the aqueous injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified. In the case that the pharmaceutical product of the present disclosure is a lyophilized injection, a required amount of the lyophilized injection dissolved in advance in water for injection can be preferably diluted with a suitable diluent and then given as an intravenous infusion. Examples of the diluent can include dextrose solution and physiological saline, dextrose solution can be preferably exemplified, and 5% dextrose solution can be more preferably exemplified.

Examples of the administration route applicable to administration of the pharmaceutical product of the present disclosure can include intravenous, intradermal, subcutaneous, intramuscular, and intraperitoneal routes, and intravenous routes are preferred.

The size of the dose required for the therapeutic treatment of a particular disease state will necessarily be varied depending on the subject treated, the route of administration and the severity of the illness being treated. For further information on routes of administration and dosage regimes, reference may be made to Chapter 25.3 in Volume 5 of Comprehensive Medicinal Chemistry (Corwin Hansch; Chairman of Editorial Board), Pergamon Press 1990.

The anti-TROP2 antibody-drug conjugate used in the present disclosure can be administered to a human once at intervals of 1 to 180 days, and can be preferably administered once a week, once every 2 weeks, once every 3 weeks, or once every 4 weeks, and can be even more preferably administered once every 3 weeks. Also, the antibody-drug conjugate used in the present invention can be administered at a dose of about 0.001 to 100 mg/kg, and can be preferably administered at a dose of 0.8 to 12.4 mg/kg. For example, the anti-TROP2 antibody-drug conjugate can be administered once every 3 weeks at a dose of 0.27 mg/kg, 0.5 mg/kg, 1.0 mg/kg, 2.0 mg/kg, 4.0 mg/kg, 6.0 mg/kg, or 8.0 mg/kg, and can be preferably administered once every 3 weeks at a dose of 4.0 or 6.0 mg/kg.

The anti-HER2 antibody-drug conjugate used in the present disclosure can be administered to a human with intervals of 1 to 180 days, can be preferably administered with intervals of a week, two weeks, three weeks, or four weeks, and can be more preferably administered with intervals of three weeks. The anti-HER2 antibody-drug conjugate used in the present disclosure can be administered in a dose of about 0.001 to 100 mg/kg per administration, and can be preferably administered in a dose of 0.8 to 12.4 mg/kg per administration. For example, the anti-HER2 antibody-drug conjugate can be administered once every three weeks at a dose of 0.8 mg/kg, 1.6 mg/kg, 3.2 mg/kg, 5.4 mg/kg, 6.4 mg/kg, 7.4 mg/kg, or 8 mg/kg, and can be preferably administered once every three weeks at a dose of 5.4 mg/kg or 6.4 mg/kg.

The bispecific checkpoint inhibitor may be administered in a suitable dose by any suitable route of administration.

In some aspects, MEDI5752 or an antigen-binding fragment thereof is administered to the subject in a dose of about 100 mg to about 1500 mg. In some aspects, the dose for administration is about 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1120 mg, about 1130 mg, about 1140 mg, about 1150 mg, about 1160 mg, about 1170 mg, about 1180 mg, about 1190 mg, about 1200 mg, about 1250 mg, about 1300 mg, about 1350 mg, about 1400 mg, about 1450 mg, or about 1500 mg.

In some aspects, a dose of MEDI5752 or an antigen-binding fragment thereof is administered to the subject once per treatment cycle. In some aspects, a treatment cycle is three weeks. In some aspects, a dose of MEDI5752 or an antigen-binding fragment thereof is administered every three weeks for about 12 months, about 24 months, about 36 months, or about 48 months.

In some aspects, the bispecific antibody or antigen-binding fragment thereof is administered in combination with one or more chemotherapeutic agents. In some aspects, the chemotherapeutic agent is carboplatin. In some aspects, the chemotherapeutic agent is a fluoropyrimidine (e.g., fluorouracil, 5-FU). In some aspects, the chemotherapeutic agent is pemetrexed. In some aspects, the chemotherapeutic agents is axitinib.

In some aspects, the bispecific checkpoint inhibitors such binding proteins disclosed herein may be formulated with a pharmaceutically acceptable carrier, excipient, or stabilizer, as pharmaceutical compositions. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy”, 21st ed., Lippincott Williams & Wilkins, (2005), and in the “Physician's Desk Reference”, 60th ed., Medical Economics, Montvale, N.J. (2005). Pharmaceutically acceptable carriers can be selected that are suitable for the mode of administration, solubility, and/or stability desired or required.

In some aspects, therapeutic compositions can be formulated for particular routes of administration, such as oral, nasal, pulmonary, topical (including buccal and sublingual), rectal, vaginal, and/or parenteral administration. The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Formulations of the disclosure that are suitable for topical or transdermal administration include powders, sprays, ointments, pastes, creams, lotions, gels, solutions, patches, and inhalants. The antibodies and other actives may be mixed under sterile conditions with a pharmaceutically acceptable carrier, and with any preservatives, buffers, or propellants which may be required (see, e.g., U.S. Pat. Nos. 7,378,110; 7,258,873; and 7,135,180; U.S. Patent Application Publication Nos. 2004/0042972 and 2004/0042971).

The formulations can be presented in unit dosage form and can be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. These dosages may be administered daily, weekly, biweekly, monthly, or less frequently, for example, biannually, depending on dosage, method of administration, disorder or symptoms to be treated, and individual subject characteristics. Dosages can also be administered via continuous infusion (such as through a pump). The administered dose may also depend on the route of administration. For example, subcutaneous administration may require a higher dosage than intravenous administration. As noted above, any commonly used dosing regimen (e.g., 1-10 mg/kg administered by injection or infusion daily or twice a week) may be adapted and suitable in the methods relating to treating human cancer patients.

### EXAMPLES

The present disclosure is specifically described in view of the examples shown below. However, the present disclosure is not limited to these. Further, it is by no means to be interpreted in a limited way.

### Example 1a: Production of Anti-TROP2 Antibody-Drug Conjugate

In accordance with a production method described in WO 2015/098099 and WO 2017/002776 and using an anti-TROP2 antibody (an antibody comprising a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]), an anti-TROP2 antibody-drug conjugate in which a drug-linker represented by the following formula:

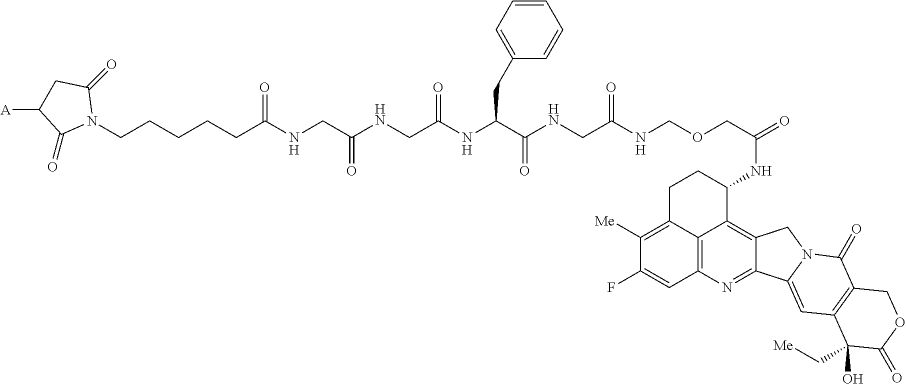

wherein A represents the connecting position to an antibody, is conjugated to the anti-TROP2 antibody via a thioether bond was produced (DS-1062: datopotamab deruxtecan). The DAR of the antibody-drug conjugate is ˜4.

### Example 1B: Production of Anti-HER2 Antibody-Drug Conjugate

In accordance with a production method described in WO2015/115091 and using an anti-HER2 antibody (an antibody comprising a heavy chain consisting of an amino acid sequence represented by SEQ ID NO: 24 (amino acid residues 1 to 449 of SEQ ID NO: 14) and a light chain consisting of an amino acid sequence consisting of all amino acid residues 1 to 214 of SEQ ID NO: 15), an anti-HER2 antibody-drug conjugate in which a drug-linker represented by the following formula:

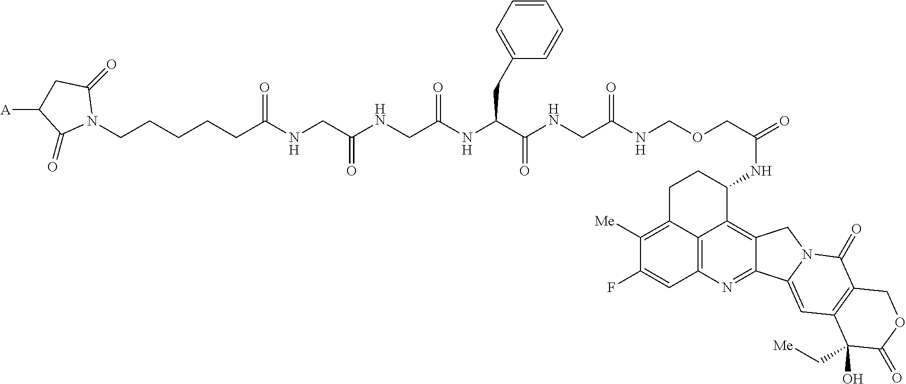

wherein A represents the connecting position to an antibody,
is conjugated to the anti-HER2 antibody via a thioether bond was produced (DS-8201: trastuzumab deruxtecan). The DAR of the antibody-drug conjugate is 7.7 or 7.8.

### Example 2a: Anti-PD-1/TIGIT Bispecific Antibody (Bispecific Checkpoint Inhibitor)

An anti-PD-1/TIGIT bispecific antibody (monovalent, humanized, IgG1 monoclonal) is created, having a first binding domain that specifically binds to PD-1, comprising a heavy chain having the amino acid sequence of SEQ ID NO: 32 and a light chain having the amino acid sequence of SEQ ID NO: 34, and a second binding domain that specifically binds TIGIT, comprising a heavy chain variable domain having the amino acid sequence of SEQ ID NO: 42 and a light chain variable domain having the amino acid sequence of SEQ ID NO: 44. The antibody is engineered to have a triple mutation L234F/L235E/P331S within its Fc domain to diminish Fc-mediated effector functionality.

### Example 2B: Anti-PD-1/CTLA-4 Bispecific Antibody (Bispecific Checkpoint Inhibitor)

An anti-PD-1/CTLA-4 bispecific antibody MEDI5752 as disclosed in U.S. Pat. No. 10,457,732 is created. MEDI5752 has a first binding domain that specifically binds to PD-1, comprising a heavy chain having the amino acid sequence of SEQ ID NO: 46 and a light chain having the amino acid sequence of SEQ ID NO: 45, and a second binding domain that specifically binds to CTLA-4, comprising a heavy chain variable domain having the amino acid sequence of SEQ ID NO: 48 and a light chain variable domain having the amino acid sequence of SEQ ID NO: 47.

### Example 3: Antitumor Test

Combination of Antibody-Drug Conjugate DS-8201 with Anti-PD-L1+Anti-CTLA-4 or with Anti-PD-1/TIGIT DuetMab

### (i) Methods:

### Tumor Models

Female BALB/c mice aged 7-9 weeks were purchased from Envigo and allowed at least 7 days acclimatisation before entry into the study. For tumor implantation, mice were shaved on the right flank and subcutaneously injected with 100 μL of cells containing 5×10⁶ EMT6 human HER2 (hHER2) cells. Tumor volume was measured three times per week using electronic callipers and calculated using the formula (width²×length)/2.

7 days post cell implant, when tumors reached approximately 170 mm³, similar-sized tumors were randomly assigned to treatment groups as shown in Table 1. Mice were euthanized when they reached humane welfare limits pertaining to tumor volume (average diameter of 15 mm) or tumor condition (ulceration of the skin above the tumor).

Humanized female NSG mice were engrafted with CD34+ cord blood stem cells at Jackson laboratory using 3 different donors. Approximately 15 weeks post engraftment, le6 Caki-1 cells were injected subcutaneously into the right flank. Tumor volumes were measured twice weekly using calipers and calculated using the formula (width²×length)/2. Mice were randomized into treatment groups as shown in Table 2 when tumors reached an average volume of 115 mm³. Mice were randomized by both tumor volume and cord blood donor to ensure 3 donors per group.

TABLE 1

| | | | Dosing schedule |
|---|---|---|---|
| | Dose | Route of | (days post cell |
| Treatment | (mg/kg) | administration | implant) |
| Vehicle | — | IV | 7, 14 |
| Vehicle | — | IP | 7, 10, 14, 17 |
| DS-8201 | 10 | IV | 7, 14 |
| Vehicle | — | IP | 7, 10, 14, 17 |
| Vehicle | — | IV | 7, 14 |
| Anti-PD-L1 | 10 | IP | 7, 10, 14, 17 |
| Vehicle | — | IV | 7, 14 |
| Anti-PD-L1 | 10 | IP | 7, 10, 14, 17 |
| Anti-CTLA-4 | 10 | IP | 7, 10, 14, 17 |
| Vehicle | — | IV | 7, 14 |
| Anti-PD-1/TIGIT | 10 | IP | 7, 10, 14, 17 |
| DuetMab | | | |
| DS-8201 | 10 | IV | 7, 14 |
| Anti-PD-L1 | 10 | IP | 7, 10, 14, 17 |
| DS-8201 | 10 | IV | 7, 14 |
| Anti-PD-L1 | 10 | IP | 7, 10, 14, 17 |
| Anti-CTLA-4 | 10 | IP | 7, 10, 14, 17 |
| DS-8201 | 10 | IV | 7, 14 |
| Anti-PD-1/TIGIT | 10 | IP | 7, 10, 14, 17 |
| DuetMab | | | |

TABLE 2

| | | | Dosing schedule |
|---|---|---|---|
| | Dose | Route of | (days post cell |
| Treatment | (mg/kg) | administration | implant) |
| Vehicle | — | IV | 29 |
| MEDI5752 | 10 | IP | 29, 32, 36, |
| | | | 39, 43, 46 |
| DS-8201 | 10 | IV | 29 |
| DS-8201 | 10 | IV | 29 |
| MEDI5752 | 10 | IP | 29, 32, 36, |
| | | | 39, 43, 46 |

### Formulation

Formulation of DS-8201 at 10 mg/Kg

Dosing solutions of DS-8201 (HA306) were prepared by diluting DS-8201 stock (20.1 mg/mL) in ABS buffer (10 mM Sodium Acetate, 10 mM Acetic Acid, 5% Sorbitol) to 2.5 mg/mL, before administration via IV injection at a dosing volume of 4 mL/kg.

Formulation of Anti-PD-L1 at 10 mg/Kg

Dosing solutions of anti-PD-L1 (clone 80, SP21-095, IgG1, D265A) were prepared by diluting stock (11.2 mg/mL) in PBS to 1 mg/mL, before administration via IP injection at a dosing volume of 10 mL/kg.

Co-Formulation of Anti-PD-L1 and Anti-CTLA-4 at 10 mg/Kg

Dosing solutions of anti-PD-L1 (clone 80, SP21-095, IgG1, D265A) and anti-CTLA-4 (SP20-103, murine IgG1) were prepared by diluting stock (anti-PD-L1-11.2 mg/mL; anti-CTLA-4-10 mg/mL) and co-formulating in PBS at 1 mg/mL of each agent, before administration via IP injection at a dosing volume of 10 mL/kg.

### Formulation of Anti-PD-1/TIGIT DuetMab

Dosing solutions of anti-PD-1/TIGIT DuetMab (mIgG2a LALA-PG) were prepared by diluting stock (C428223DEC21EO, 2.7 mg/mL) in PBS to 1 mg/mL, before administration via IP injection at a dosing volume of 10 mL/kg.

### Formulation of MEDI5752

Dosing solutions of MEDI5752 were prepared by diluting stock (Lot #ML00669-50, 63.41 mg/ml) in PBS to 2 mg/ml, before administration via IP injection at a dosing volume of 5 mL/kg.

### Fine Needle Aspirate (FNA) Sampling

Mice were anesthetized using isoflurane and placed on a rotating anaesthetic platform. A 25 gauge needle was attached to a 1 mL syringe filled with 0.9 mL of cold RPMI media. The needle was inserted horizontally into the tumor and rotated to dislodge tissue. Negative pressure was generated by pulling back the syringe plunger by approximately 100 pL to withdraw cells into the media. Roughly 200 pL of the media containing cells was then flushed out of the syringe into a 1.5 mL Eppendorf tube. This was repeated a further four times at different sites around the entire tumor to maximize the representativity of the sampling and the tube was immediately placed on ice.

### Flow Cytometric Staining

Cells were stained with a fixable viability dye (Thermo Fisher) and blocked with antibodies to CD16/CD32 (eBioscience) before staining with fluorescence-conjugated antibodies in flow cytometry staining buffer (2% bovine serum albumin, 0.1% sodium azide, 2 mM EDTA) with 50% Brilliant Stain Buffer (BD Biosciences). Intracellular staining was performed using the FoxP3/Transcription Factor Staining Buffer Set (eBioscience) and cells were fixed in 3.7% formaldehyde. Cells were acquired in flow cytometry staining buffer on a BD FACSymphony flow cytometer (BD Biosciences) and analyzed using FlowJo (TreeStar). Samples containing fewer than 1000CD45⁺ cells were excluded from analysis due to low event count.

### Statistics

Tumor growth rates were calculated for each animal based on fitting each tumor's growth curve (day 0-15 post initiation of treatment) to an exponential model log 10(tumorvolume)=a+b·time+error, where a and b are parameters that correspond to the log initial volume and growth rate, respectively. Statistical significance of differences in tumor growth rates between groups was evaluated using a Mann Whitney U test. Box plots show median and 25^{th}-75^{th} quartiles, whiskers show minimum and maximum.

For flow cytometry data of cell frequencies, data was processed with a Beta Regression treating treatment as a fixed effect. p-values are reported as follows; p≥0.05 (ns, not significant); *, p<0.05; **, p<0.01; ***, p<0.001; ****, p<0.0001.

### (ii) Tests:

EMT6 hHER2-tumor bearing mice were treated with combinations of DS-8201 and Immuno-Oncology (IO) agents, i.e. treated with 10 mg/kg DS-8201 alone, or in combination with 10 mg/kg anti-PD-L1, 10 mg/kg anti-PD-L1+10 mg/kg anti-CTLA-4 and 10 mg/kg anti-PD-1/TIGIT DuetMab according to dosing schedule outlined in Table 1. Results are shown in FIGS. 59 and 60, and in Tables 3 and 4.

FNA tumor samples were taken 8 days post treatment initiation, from EMT6 hHER2-tumor bearing mice treated with combinations of DS-8201 alone, or in combination with 10 mg/kg anti-PD-1/TIGIT DuetMab according to dosing schedule outlined in Table 1, and pharmacodynamic changes assessed via flow cytometry. Samples with fewer than 1000 CD45+ cells were excluded from analysis (n=7-9 FNA samples per group). T cells=CD45+CD3+ cells. Tregs=CD45+ CD3+ CD4+ FoxP3+ cells. NK cells=CD45+CD3-NKp46+ cells. Results are shown in FIG. 61. In a similar study, tumor samples were taken 10 days post treatment initiation, from EMT6 hHER2-tumor bearing mice treated with vehicle or DS-8201 alone, and pharmacodynamic changes in CD8+ T cell CTLA-4 expression were assessed via flow cytometry (n=8 samples per group). Results are shown in FIG. 62.

In a separate study, Caki-1-tumor bearing mice were treated with combinations of DS-8201 and/or the IO agent MEDI5752 according to the dosing schedule outlined in Table 2. Results are shown in FIG. 63.

Peripheral blood was sampled on day 22 post treatment initiation in the Caki-1 study and analyzed via flow cytometry for human specific T cell activation markers (n=5 samples per group). Results are shown in FIG. 64.

(iii) Results:

FIGS. 59 and 60 show anti-tumor efficacy and tumor growth rates, respectively, of the DS-8201 combinations with the IO agents in the EMT6 hHER2-tumor bearing mice. In FIG. 59, graphs A show mean changes in tumor volume over time. Data shown are mean±SEM (n=10 mice per treatment group). In FIG. 59, graphs B show changes in tumor volume over time for individual mice (CR=complete response). In FIG. 60, data shown are tumor growth rates (n=10 mice per treatment group).

Single agent DS-8201 treatment significantly delayed tumor growth (53.2% growth rate inhibition, p=0.0002), as did anti-PD-L1 (46.2% growth rate inhibition, p=0.0185), anti-PD-L1+anti-CTLA-4 (86.2% growth rate inhibition, p=0.0355) and anti-PD-1/TIGIT DuetMab (120.3% growth rate inhibition, p<0.0001) (FIG. 59 graphs A, FIG. 60, Table 3).

TABLE 3

| | | Growth | p-value | |
|---|---|---|---|---|
| | | rate | (Mann- | |
| | | inhibition | Whitney | |
| | Growth | versus | U test | Complete |
| | rate | Vehicle | versus | responses |
| Treatment group | median | (%) | Vehicle) | (%) |
| Vehicle | 0.0558 | 0.0 | N/A | N/A |
| DS-8201 | 0.0248 | 53.2 | 0.0002 | 0 |
| Anti-PD-L1 | 0.0391 | 46.2 | 0.0185 | 10 |
| Anti-PD-L1 | 0.0303 | 86.2 | 0.0355 | 30 |
| Anti-CTLA-4 | | | | |
| Anti-PD-1/TIGIT | −0.0008 | 120.3 | <0.0001 | 30 |
| DuetMab | | | | |

Tumor growth inhibition was further enhanced over DS-8201 monotherapy when DS-8201 was combined with anti-PD-L1 (117.4% growth rate inhibition, p=0.0007), anti-PD-L1+anti-CTLA-4 (253.5% growth rate inhibition, p=0.0002) and anti-PD-1 TIGIT DuetMab (273.6% growth rate inhibition, p<0.0001) (FIG. 59 graphs A, Table 4). Combination of DS-8201 with anti-PD-L1+anti-CTLA-4 and anti-PD-1/TIGIT DuetMab resulted in complete responses in 80% and 60% of animals respectively, whilst combination with anti-PD-L1 resulted in complete responses in just 10% of animals (FIG. 59 graphs B, Table 4).

TABLE 4

| | | Growth | p-value | |
|---|---|---|---|---|
| | | rate | (Mann- | |
| | | inhibition | Whitney | |
| | Growth | versus | U test | Complete |
| | rate | Vehicle | versus | responses |
| Treatment group | median | (%) | Vehicle) | (%) |
| DS-8201 | 0.0022 | 117.4 | 0.0007 | 10 |
| Anti-PD-L1 | | | | |
| DS-8201 | −0.0426 | 253.5 | 0.0002 | 80 |
| Anti-PD-L1 | | | | |
| Anti-CTLA-4 | | | | |
| DS-8201 | −0.0563 | 273.6 | <0.0001 | 60 |
| Anti-PD-1/TIGIT | | | | |
| DuetMab | | | | |

As regards pharmacodynamic changes, the FNA tumor samples taken 8 days post DS-8201 treatment initiation revealed increased tumoral proportion of total immune cells (CD45+), Tregs and NK cells, and increased the proportion of T cells expressing PD-1, Ki67 and TIGIT. Combination of DS-8201 with anti-PD-1/TIGIT DuetMab treatment further increased tumoral T cell (% live) and NK cell (% CD45+) content over monotherapies, and also increased the proportion of CD8+ T cells (% CD3+) (FIG. 61). In a similar study, DS-8201 monotherapy treatment significantly increased expression of CTLA-4 on tumoral CD8+ T cells (FIG. 62).

In a CD34+ hematopoietic stem cell engrafted humanized mouse model bearing renal Caki-1 tumors, the combination of DS-8201 and MEDI5752 resulted in greater tumor growth inhibition than either monotherapy treatment alone, and was significantly better than DS-8201 (FIG. 63). Relative to the DS-8201 treatment groups, mice receiving MEDI5752 monotherapy exhibited substantial body weight loss, which resulted in early termination of this treatment group at Day 50 post tumor implantation. Interestingly, these effects were not observed in the group receiving MEDI5752 in combination with DS-8201.

Peripheral blood flow analysis was performed on day 22 post treatment in the Caki-1 study and revealed increased proliferating CD4+ and CD8+ T cells as well as increased granzyme B+ CD8 and ICOS+ CD4 cells. These effects were observed with monotherapy MEDI5752 and maintained in the combination treatment groups (FIG. 64).

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiments may be practiced in many ways and the claims include any equivalents thereof.

### Free Text of Sequence Listing

SEQ ID NO: 1—Amino acid sequence of a heavy chain of anti-TROP2 antibody
SEQ ID NO: 2—Amino acid sequence of a light chain of anti-TROP2 antibody
SEQ ID NO: 3—Amino acid sequence of a heavy chain CDRH1 [=amino acid residues 50 to 54 of SEQ ID NO: 1]
SEQ ID NO: 4—Amino acid sequence of a heavy chain CDRH2 [=amino acid residues 69 to 85 of SEQ ID NO: 1]
SEQ ID NO: 5—Amino acid sequence of a heavy chain CDRH3 [=amino acid residues 118 to 129 of SEQ ID NO: 1]
SEQ ID NO: 6—Amino acid sequence of a light chain CDRL1 [=amino acid residues 44 to 54 of SEQ ID NO: 2]
SEQ ID NO: 7—Amino acid sequence of a light chain CDRL2 [=amino acid residues 70 to 76 of SEQ ID NO: 2]
SEQ ID NO: 8—Amino acid sequence of a light chain CDRL3 [=amino acid residues 109 to 117 of SEQ ID NO: 2]
SEQ ID NO: 9—Amino acid sequence of a heavy chain variable region [=amino acid residues 20 to 140 of SEQ ID NO: 1]
SEQ ID NO: 10—Amino acid sequence of a light chain variable region [=amino acid residues 21 to 129 of SEQ ID NO: 2]
SEQ ID NO: 11—Amino acid sequence of a heavy chain [=amino acid residues 20 to 469 of SEQ ID NO: 1]
SEQ ID NO: 12—Amino acid sequence of a heavy chain [=amino acid residues 20 to 470 of SEQ ID NO: 1]
SEQ ID NO: 13—Amino acid sequence of a light chain [=amino acid residues 21 to 234 of SEQ ID NO: 2]
SEQ ID NO: 14—Amino acid sequence of a heavy chain of an anti-HER2 antibody
SEQ ID NO: 15—Amino acid sequence of a light chain of an anti-HER2 antibody
SEQ ID NO: 16—Amino acid sequence of a heavy chain CDRH1 [=amino acid residues 26 to 33 of SEQ ID NO: 14]
SEQ ID NO: 17—Amino acid sequence of a heavy chain CDRH2 [=amino acid residues 51 to 58 of SEQ ID NO: 14]
SEQ ID NO: 18—Amino acid sequence of a heavy chain CDRH3 [=amino acid residues 97 to 109 of SEQ ID NO: 14]
SEQ ID NO: 19—Amino acid sequence of a light chain CDRL1 [=amino acid residues 27 to 32 of SEQ ID NO: 15]
SEQ ID NO: 20—Amino acid sequence comprising amino acid sequence of a light chain CDRL2 (SAS) [=amino acid residues 50 to 56 of SEQ ID NO: 15]
SEQ ID NO: 21—Amino acid sequence of a light chain CDRL3 [=amino acid residues 89 to 97 of SEQ ID NO: 15]
SEQ ID NO: 22—Amino acid sequence of a heavy chain variable region [=amino acid residues 1 to 120 of SEQ ID NO: 14]
SEQ ID NO: 23—Amino acid sequence of a light chain variable region [=amino acid residues 1 to 107 of SEQ ID NO: 15]
SEQ ID NO: 24—Amino acid sequence of a heavy chain [=amino acid residues 1 to 449 of SEQ ID NO: 14]
SEQ ID NO: 25—Amino acid sequence of an anti-PD1 heavy chain CDRH1 of anti-PD-1/TIGIT bispecific antibody
SEQ ID NO: 26—Amino acid sequence of an anti-PD1 heavy chain CDRH2 of anti-PD-1/TIGIT bispecific antibody
SEQ ID NO: 27—Amino acid sequence of an anti-PD1 heavy chain CDRH3 of anti-PD-1/TIGIT bispecific antibody
SEQ ID NO: 28—Amino acid sequence of an anti-PD1 light chain CDRL1 of anti-PD-1/TIGIT bispecific antibody
SEQ ID NO: 29—Amino acid sequence of an anti-PD1 light chain CDRL2 of anti-PD-1/TIGIT bispecific antibody
SEQ ID NO: 30—Amino acid sequence of an anti-PD1 light chain CDRL3 of anti-PD-1/TIGIT bispecific antibody
SEQ ID NO: 31—Amino acid sequence of an anti-PD1 heavy chain variable region of anti-PD-1/TIGIT bispecific antibody
SEQ ID NO: 32—Amino acid sequence of an anti-PD1 heavy chain of anti-PD-1/TIGIT bispecific antibody
SEQ ID NO: 33—Amino acid sequence of an anti-PD1 light chain variable region of anti-PD-1/TIGIT bispecific antibody
SEQ ID NO: 34—Amino acid sequence of an anti-PD1 light chain of anti-PD-1/TIGIT bispecific antibody
SEQ ID NO: 35—Amino acid sequence of an anti-TIGIT heavy chain CDRH1 of anti-PD-1/TIGIT bispecific antibody
SEQ ID NO: 36—Amino acid sequence of an anti-TIGIT heavy chain CDRH2 of anti-PD-1/TIGIT bispecific antibody
SEQ ID NO: 37—Amino acid sequence of an anti-TIGIT heavy chain CDRH3 of anti-PD-1/TIGIT bispecific antibody
SEQ ID NO: 38—Amino acid sequence of an anti-TIGIT light chain CDRL1 of anti-PD-1/TIGIT bispecific antibody
SEQ ID NO: 39—Amino acid sequence of an anti-TIGIT light chain CDRL2 of anti-PD-1/TIGIT bispecific antibody
SEQ ID NO: 40—Amino acid sequence of an anti-TIGIT light chain CDRL3 of anti-PD-1/TIGIT bispecific antibody
SEQ ID NO: 41—Amino acid sequence of an anti-TIGIT heavy chain variable region of anti-PD-1/TIGIT bispecific antibody
SEQ ID NO: 42—Amino acid sequence of an anti-TIGIT heavy chain of anti-PD-1/TIGIT bispecific antibody
SEQ ID NO: 43—Amino acid sequence of an anti-TIGIT light chain variable region of anti-PD-1/TIGIT bispecific antibody
SEQ ID NO: 44—Amino acid sequence of an anti-TIGIT light chain of anti-PD-1/TIGIT bispecific antibody
SEQ ID NO: 45—Amino acid sequence of an anti-PD1 light chain of anti-PD-1/CTRL-4 bispecific antibody
SEQ ID NO: 46—Amino acid sequence of an anti-PD1 heavy chain of anti-PD-1/CTRL-4 bispecific antibody
SEQ ID NO: 47—Amino acid sequence of an anti-CTLA-4 light chain of anti-PD-1/CTRL-4 bispecific antibody
SEQ ID NO: 48—Amino acid sequence of an anti-CTLA-4 heavy chain of anti-PD-1/CTRL-4 bispecific antibody
SEQ ID NO: 49—Amino acid sequence of an anti-PD1 light chain CDRL1 of anti-PD-1/CTRL-4 bispecific antibody
SEQ ID NO: 50—Amino acid sequence of an anti-PD1 light chain CDRL2 of anti-PD-1/CTRL-4 bispecific antibody
SEQ ID NO: 51—Amino acid sequence of an anti-PD1 light chain CDRL3 of anti-PD-1/CTRL-4 bispecific antibody
SEQ ID NO: 52—Amino acid sequence of an anti-PD1 heavy chain CDRH1 of anti-PD-1/CTRL-4 bispecific antibody
SEQ ID NO: 53—Amino acid sequence of an anti-PD1 heavy chain CDRH2 of anti-PD-1/CTRL-4 bispecific antibody
SEQ ID NO: 54—Amino acid sequence of an anti-PD1 heavy chain CDRH3 of anti-PD-1/CTRL-4 bispecific antibody
SEQ ID NO: 55—Amino acid sequence of an anti-CTLA-4 light chain CDRL1 of anti-PD-1/CTRL-4 bispecific antibody
SEQ ID NO: 56—Amino acid sequence of an anti-CTLA-4 light chain CDRL2 of anti-PD-1/CTRL-4 bispecific antibody
SEQ ID NO: 57—Amino acid sequence of an anti-CTLA-4 light chain CDRL3 of anti-PD-1/CTRL-4 bispecific antibody
SEQ ID NO: 58—Amino acid sequence of an anti-CTLA-4 heavy chain CDRH1 of anti-PD-1/CTRL-4 bispecific antibody
SEQ ID NO: 59—Amino acid sequence of an anti-CTLA-4 heavy chain CDRH2 of anti-PD-1/CTRL-4 bispecific antibody
SEQ ID NO: 60—Amino acid sequence of an anti-CTLA-4 heavy chain CDRH3 of anti-PD-1/CTRL-4 bispecific antibody

1. A pharmaceutical product comprising an antibody-drug conjugate and a bispecific checkpoint inhibitor for administration in combination, wherein the antibody-drug conjugate is an antibody-drug conjugate in which a drug-linker represented by the following formula: 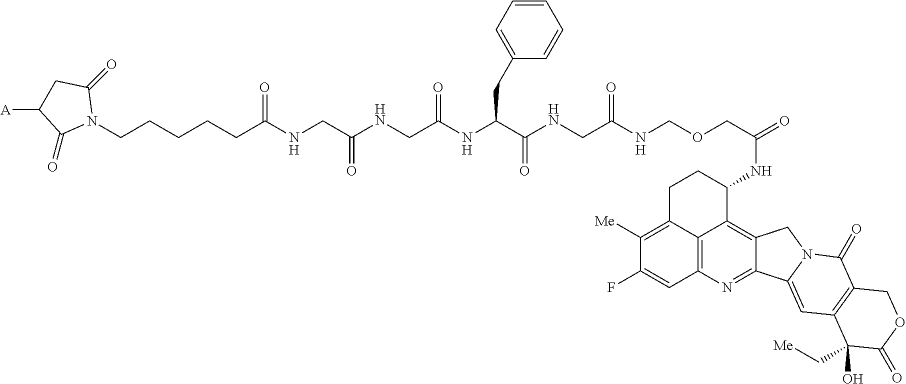
wherein A represents the connecting position to an antibody, is conjugated to an antibody, preferably an anti-TROP2 or anti-HER2 antibody, via a thioether bond.

2. The pharmaceutical product according to claim 1, wherein the drug-linker is conjugated to an anti-TROP2 antibody.

3. The pharmaceutical product according to claim 2, wherein the anti-TROP2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence represented by SEQ ID NO: 7 and CDRL3 consisting of an amino acid sequence represented by SEQ ID NO: 8.

4. The pharmaceutical product according to claim 3, wherein the anti-TROP2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10.

5. The pharmaceutical product according to claim 4, wherein the anti-TROP2 antibody is an antibody comprising a heavy chain consisting of an amino acid sequence represented by SEQ ID NO: 12 and a light chain consisting of an amino acid sequence represented by SEQ ID NO: 13.

6. The pharmaceutical product according to claim 4, wherein the anti-TROP2 antibody is an antibody comprising a heavy chain consisting of an amino acid sequence represented by SEQ ID NO: 11 and a light chain consisting of an amino acid sequence represented by SEQ ID NO: 13.

7. The pharmaceutical product according to any one of claims 2 to 6, wherein the average number of units of the drug-linker conjugated per anti-TROP2 antibody molecule in the antibody-drug conjugate is in the range of from 3.5 to 4.5.

8. The pharmaceutical product according to claim 7, wherein the anti-TROP2 antibody-drug conjugate is datopotamab deruxtecan (DS-1062).

9. The pharmaceutical product according to claim 1, wherein the drug-linker is conjugated to an anti-HER2 antibody.

10. The pharmaceutical product according to claim 9, wherein the anti-HER2 antibody is an antibody comprising a heavy chain comprising CDRH1 consisting of an amino acid sequence represented by SEQ ID NO: 16, CDRH2 consisting of an amino acid sequence represented by SEQ ID NO: 17 and CDRH3 consisting of an amino acid sequence represented by SEQ ID NO: 18, and a light chain comprising CDRL1 consisting of an amino acid sequence represented by SEQ ID NO: 19, CDRL2 consisting of an amino acid sequence consisting of amino acid residues 1 to 3 of SEQ ID NO: 20 and CDRL3 consisting of an amino acid sequence represented by SEQ ID NO: 21.

11. The pharmaceutical product according to claim 10, wherein the anti-HER2 antibody is an antibody comprising a heavy chain comprising a heavy chain variable region consisting of an amino acid sequence represented by SEQ ID NO: 22 and a light chain comprising a light chain variable region consisting of an amino acid sequence represented by SEQ ID NO: 23.

12. The pharmaceutical product according to claim 11, wherein the anti-HER2 antibody is an antibody comprising a heavy chain consisting of an amino acid sequence represented by SEQ ID NO: 14 and a light chain consisting of an amino acid sequence represented by SEQ ID NO: 15.

13. The pharmaceutical product according to claim 11, wherein the anti-HER2 antibody is an antibody comprising a heavy chain consisting of an amino acid sequence represented by SEQ ID NO: 24 and a light chain consisting of an amino acid sequence represented by SEQ ID NO: 15.

14. The pharmaceutical product according to any one of claims 9 to 13, wherein the average number of units of the drug-linker conjugated per anti-HER2 antibody molecule in the antibody-drug conjugate is in the range of from 7 to 8.

15. The pharmaceutical product according to claim 14, wherein the anti-HER2 antibody-drug conjugate is trastuzumab deruxtecan (DS-8201).

16. The pharmaceutical product according to any one of claims 1 to 15, wherein the bispecific checkpoint inhibitor is a bispecific binding protein that comprises a first binding domain that specifically binds to PD-1, and a second binding domain that specifically binds to CTLA-4 or TIGIT.

17. The pharmaceutical product according to claim 16, wherein the bispecific binding protein comprises:
a) a first binding domain that specifically binds to PD-1, wherein the first binding domain comprises a heavy chain variable domain comprising a CDRH1 having the amino acid sequence of SEQ ID NO: 25, a CDRH2 having the amino acid sequence of SEQ ID NO: 26, and a CDRH3 having the amino acid sequence of SEQ ID NO: 27, and a light chain variable domain comprising a CDRL1 having the amino acid sequence of SEQ ID NO: 28, a CDRL2 having the amino acid sequence of SEQ ID NO: 29 and a CDRL3 having the amino acid sequence of SEQ ID NO: 30; and
b) a second binding domain that specifically binds to TIGIT, wherein the second binding domain comprises a heavy chain variable domain comprising a CDRH1 having the amino acid sequence of SEQ ID NO: 35, a CDRH2 having the amino acid sequence of SEQ ID NO: 36, and a CDRH3 having the amino acid sequence of SEQ ID NO: 37, and a light chain variable domain comprising a CDRL1 having the amino acid sequence of SEQ ID NO: 38, a CDRL2 having the amino acid sequence of SEQ ID NO: 39, and a CDRL3 having the amino acid sequence of SEQ ID NO: 40.

18. The pharmaceutical product according to claim 17, wherein the first binding domain that specifically binds to PD-1 comprises a heavy chain variable domain having the amino acid sequence of SEQ ID NO: 31 and a light chain variable domain having the amino acid sequence of SEQ ID NO: 33.

19. The pharmaceutical product according to claim 17, wherein first binding domain that specifically binds to PD-1 comprises a heavy chain variable domain having an amino acid sequence that is at least 90% identical to the amino acid sequence of SEQ ID NO: 31 and a light chain variable domain having an amino acid sequence that is at least 90% identical to the amino acid sequence of SEQ ID NO: 33.

20. The pharmaceutical product according to any one of claims 17 to 19, wherein the first binding domain that specifically binds to PD-1 comprises a heavy chain having the amino acid sequence of SEQ ID NO: 32 and a light chain having the amino acid sequence of SEQ ID NO: 34.

21. The pharmaceutical product according to any one of claims 17 to 19, wherein first binding domain that specifically binds to PD-1 comprises a heavy chain having an amino acid sequence that is at least 90% identical to the amino acid sequence of SEQ ID NO: 32 and a light chain having an amino acid sequence that is at least 90% identical to the amino acid sequence of SEQ ID NO: 34.

22. The pharmaceutical product according to any one of claims 17 to 21, wherein the second binding domain that specifically binds TIGIT comprises a heavy chain variable domain having the amino acid sequence of SEQ ID NO: 41 and a light chain variable domain having the amino acid sequence of SEQ ID NO: 43.

23. The pharmaceutical product according to any one of claims 17 to 21, wherein the second binding domain that specifically binds to TIGIT comprises a heavy chain variable domain having an amino acid sequence that is at least 90% identical to the amino acid sequence of SEQ ID NO: 41 and a light chain variable domain having an amino acid sequence that is at least 90% identical to the amino acid sequence of SEQ ID NO: 43.

24. The pharmaceutical product according to any one of claims 17 to 23, wherein the second binding domain that specifically binds to TIGIT comprises a heavy chain having the amino sequence of SEQ ID NO: 42 and a light chain having the amino acid sequence of SEQ ID NO: 44.

25. The pharmaceutical product according to any one of claims 17 to 23, wherein the second binding domain that specifically binds to TIGIT comprises a heavy chain having an amino acid sequence that is at least 90% identical to the amino acid sequence of SEQ ID NO: 42 and a light chain having an amino acid sequence that is at least 90% identical to the amino acid sequence of SEQ ID NO: 44.

26. The pharmaceutical product according to claim 16, wherein the bispecific binding protein comprises:
a) a first binding domain that specifically binds to PD-1, wherein the first binding domain comprises a heavy chain variable domain comprising a CDRH1 having the amino acid sequence of SEQ ID NO: 52, a CDRH2 having the amino acid sequence of SEQ ID NO: 53, and a CDRH3 having the amino acid sequence of SEQ ID NO: 54, and a light chain variable domain comprising a CDRL1 having the amino acid sequence of SEQ ID NO: 49, a CDRL2 having the amino acid sequence of SEQ ID NO: 50 and a CDRL3 having the amino acid sequence of SEQ ID NO: 51; and
b) a second binding domain that specifically binds to CTLA-4, wherein the second binding domain comprises a heavy chain variable domain comprising a CDRH1 having the amino acid sequence of SEQ ID NO: 58, a CDRH2 having the amino acid sequence of SEQ ID NO: 59, and a CDRH3 having the amino acid sequence of SEQ ID NO: 60, and a light chain variable domain comprising a CDRL1 having the amino acid sequence of SEQ ID NO: 55, a CDRL2 having the amino acid sequence of SEQ ID NO: 56, and a CDRL3 having the amino acid sequence of SEQ ID NO: 57.

27. The pharmaceutical product according to claim 26, wherein the first binding domain that specifically binds to PD-1 comprises a heavy chain having the amino acid sequence of SEQ ID NO: 46 and a light chain having the amino acid sequence of SEQ ID NO: 45.

28. The pharmaceutical product according to claim 26, wherein first binding domain that specifically binds to PD-1 comprises a heavy chain having an amino acid sequence that is at least 90% identical to the amino acid sequence of SEQ ID NO: 46 and a light chain having an amino acid sequence that is at least 90% identical to the amino acid sequence of SEQ ID NO: 45.

29. The pharmaceutical product according to any one of claims 26 to 28, wherein the second binding domain that specifically binds to CTLA-4 comprises a heavy chain having the amino sequence of SEQ ID NO: 48 and a light chain having the amino acid sequence of SEQ ID NO: 47.

30. The pharmaceutical product according to any one of claims 26 to 28, wherein the second binding domain that specifically binds to CTLA-4 comprises a heavy chain having an amino acid sequence that is at least 90% identical to the amino acid sequence of SEQ ID NO: 48 and a light chain having an amino acid sequence that is at least 90% identical to the amino acid sequence of SEQ ID NO: 47.

31. The pharmaceutical product according to claim 20 or 27, wherein the light chain constant region is a kappa chain.

32. The pharmaceutical product according to claim 24 or 29, wherein the light chain constant region is a lambda chain.

33. The pharmaceutical product according to any one of claims 16 to 32, wherein the binding protein is an antibody.

34. The pharmaceutical product according to claim 33, wherein the antibody is an IgG antibody.

35. The pharmaceutical product according to claim 34, wherein the antibody is an IgG1 antibody.

36. The pharmaceutical product according to claim 34 or 35, wherein the antibody is human or humanized.

37. The pharmaceutical product according to any one of claims 33 to 36, wherein the bispecific antibody is monovalent.

38. The pharmaceutical product according to any one of claims 16 to 37, wherein the bispecific binding protein is a DuetMab.

39. The pharmaceutical product according to any one of claims 16 to 38, wherein the bispecific binding protein comprises a variant Fc region.

40. The pharmaceutical product according to claim 39, wherein the variant Fc region comprises at least one substitution selected from 221K, 221Y, 225E, 225K, 225W, 228P, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 2621, 262A, 262T, 262E, 2631, 263A, 263T, 263M, 264L, 2641, 264W, 264T, 264R, 264F, 264M, 264Y, 264E, 265A, 265G, 265N, 265Q, 265Y, 265F, 265V, 2651, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 296G, 297S, 297D, 297E, 298A, 298H, 298I, 298T, 298F, 299I, 299L, 299A, 299S, 299V, 299H, 299F, 299E, 305I, 308F, 313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 434W, 434Y, 436H, 440Y and 443W as numbered by the EU index as set forth in Kabat.

41. The pharmaceutical product according to claim 39, wherein the variant Fc region comprises one or more amino acid substitutions at positions selected from 428 and 434 as numbered by the EU index as set forth in Kabat.

42. The pharmaceutical product according to any one of claims 39 to 41, wherein the variant Fc region comprises one or more amino acid substitutions selected from 428L, 428F, 434A, 434W, and 434Y.

43. The pharmaceutical product according to any one of claims 39 to 42, wherein the variant Fc region comprises a L234F/L235E/P331S triple mutation (TM).

44. The pharmaceutical product according to any one of claims 16 to 43, wherein the bispecific binding protein comprises an Fc region that is aglycosylated.

45. The pharmaceutical product according to any one of claims 16 to 43, wherein the bispecific binding protein comprises an Fc region that is deglycosylated.

46. The pharmaceutical product according to any one of claims 16 to 43, wherein the bispecific binding protein comprises an Fc region that has reduced fucosylation or is afucosylated.

47. The pharmaceutical product according to any one of claims 1 to 46 wherein the product is a combined preparation comprising the antibody-drug conjugate and the bispecific checkpoint inhibitor, for separate simultaneous administration.

48. The pharmaceutical product according to any one of claims 1 to 46 wherein the product is a combined preparation comprising the antibody-drug conjugate and the bispecific checkpoint inhibitor, for sequential or separate simultaneous administration.

49. The pharmaceutical product according to any one of claims 1 to 48, wherein the product is for treating cancer.

50. The pharmaceutical product according to claim 49, wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, endometrial cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, melanoma, cervical cancer, uterine cancer, testicular cancer, and renal cell carcinoma.

51. The pharmaceutical product according to claim 50, wherein the cancer is breast cancer.

52. The pharmaceutical product according to claim 51, wherein the breast cancer is HER2 positive breast cancer.

53. The pharmaceutical product according to claim 51, wherein the breast cancer is HER2 low breast cancer.

54. The pharmaceutical product according to claim 51, wherein the breast cancer is triple negative breast cancer.

55. The pharmaceutical product according to claim 51, wherein the breast cancer is hormone receptor (HR)-positive, HER2-negative breast cancer.

56. The pharmaceutical product according to claim 50, wherein the cancer is lung cancer.

57. The pharmaceutical product according to claim 56, wherein the lung cancer is non-small cell lung cancer.

58. The pharmaceutical product according to claim 57, wherein the non-small cell lung cancer is non-small cell lung cancer with actionable genomic alterations.

59. The pharmaceutical product according to claim 57, wherein the non-small cell lung cancer is non-small cell lung cancer lung cancer without actionable genomic alterations.

60. The pharmaceutical product according to claim 50, wherein the cancer is colorectal cancer.

61. The pharmaceutical product according to claim 50, wherein the cancer is gastric cancer.

62. The pharmaceutical product according to claim 50, wherein the cancer is pancreatic cancer.

63. The pharmaceutical product according to claim 50, wherein the cancer is ovarian cancer.

64. The pharmaceutical product according to claim 50, wherein the cancer is prostate cancer.

65. The pharmaceutical product according to claim 50, wherein the cancer is kidney cancer.

66. The pharmaceutical product according to claim 50, wherein the cancer is bladder cancer.

67. The pharmaceutical product according to claim 50, wherein the cancer is endometrial cancer.

68. The pharmaceutical product according to claim 50, wherein the cancer is biliary tract cancer.

69. A pharmaceutical product as defined in any one of claims 1 to 48, for use in treating cancer.

70. The pharmaceutical product for the use according to claim 69, wherein the cancer is as defined in any one of claims 50 to 68.

71. The pharmaceutical product according to any one of claims 1 to 48, further comprising carboplatin for administration in combination with the antibody-drug conjugate and the bispecific checkpoint inhibitor.

72. The pharmaceutical product according to any one of claims 1 to 48, further comprising a fluoropyrimidine for administration in combination with the antibody-drug conjugate and the bispecific checkpoint inhibitor.

73. Use of an antibody-drug conjugate in the manufacture of a medicament for use in combination with a bispecific checkpoint inhibitor, wherein the antibody-drug conjugate and the bispecific checkpoint inhibitor are as defined in any one of claims 1 to 46, for treating cancer.

74. The use according to claim 73 wherein the medicament is for use in combination with the bispecific checkpoint inhibitor by sequential administration.

75. The use according to claim 73 wherein the medicament is for use in combination with the bispecific checkpoint inhibitor by separate simultaneous administration.

76. Use of a bispecific checkpoint inhibitor in the manufacture of a medicament for use in combination with an antibody-drug conjugate, wherein the antibody-drug conjugate and the bispecific checkpoint inhibitor are as defined in any one of claims 1 to 46, for treating cancer.

77. The use according to claim 76 wherein the medicament is for use in combination with the antibody-drug conjugate by sequential administration.

78. The use according to claim 76 wherein the medicament is for use in combination with the antibody-drug conjugate by separate simultaneous administration.

79. The use according to any one of claims 73 to 78, wherein the cancer is as defined in any one of claims 50 to 68.

80. An antibody-drug conjugate for use, in combination with a bispecific checkpoint inhibitor, in the treatment of cancer, wherein the antibody-drug conjugate and the bispecific checkpoint inhibitor are as defined in any one of claims 1 to 46.

81. The antibody-drug conjugate for the use according to claim 80, wherein the cancer is as defined in any one of claims 50 to 68.

82. The antibody-drug conjugate for the use according to claim 80 or 81, wherein the use comprises administration of the antibody-drug conjugate and the bispecific checkpoint inhibitor sequentially.

83. The antibody-drug conjugate for the use according to claim 80 or 81, wherein the use comprises administration of the antibody-drug conjugate and the bispecific checkpoint inhibitor separately and simultaneously.

84. An antibody-drug conjugate for use in the treatment of cancer in a subject, wherein said treatment comprises the sequential or separate simultaneous administration of i) the antibody-drug conjugate, and ii) a bispecific checkpoint inhibitor to said subject, wherein the antibody-drug conjugate and the bispecific checkpoint inhibitor are as defined in any one of claims 1 to 46.

85. A bispecific checkpoint inhibitor for use, in combination with an antibody-drug conjugate, in the treatment of cancer, wherein the antibody-drug conjugate and the bispecific checkpoint inhibitor are as defined in any one of claims 1 to 46.

86. The bispecific checkpoint inhibitor for the use according to claim 85, wherein the cancer is as defined in any one of claims 50 to 68.

87. The bispecific checkpoint inhibitor for the use according to claim 85 or 86, wherein the use comprises administration of the antibody-drug conjugate and the bispecific checkpoint inhibitor sequentially.

88. The bispecific checkpoint inhibitor for the use according to claim 85 or 86, wherein the use comprises administration of the antibody-drug conjugate and the bispecific checkpoint inhibitor separately and simultaneously.

89. A bispecific checkpoint inhibitor for use in the treatment of cancer in a subject, wherein said treatment comprises the sequential or separate simultaneous administration of i) the bispecific checkpoint inhibitor, and ii) an antibody-drug conjugate to said subject, wherein the bispecific checkpoint inhibitor and the antibody-drug conjugate are as defined in any one of claims 1 to 46.

90. A method of treating cancer comprising administering an antibody-drug conjugate and a bispecific checkpoint inhibitor as defined in any one of claims 1 to 46 in combination to a subject in need thereof.

91. The method according to claim 90, wherein the cancer is as defined in any one of claims 50 to 68.

92. The method according to claim 90 or 91, wherein the method comprises administering the antibody-drug conjugate and the bispecific checkpoint inhibitor sequentially.

93. The method according to claim 90 or 91, wherein the method comprises administering the antibody-drug conjugate and the bispecific checkpoint inhibitor separately and simultaneously.
